(19) United States
(12) Patent Application Publication
(10) Pub. No.: US 2011/0105443 A1
(43) Pub. Date: May 5, 2011

(54) SALTS OF MEMANTINE AND COX-INHIBITORS AND THEIR CRYSTAL FORM IN THE TREATMENT OF PAIN

(75) Inventors: Helmut Heinrich Buschmann, Aachen (DE); Nicolas Tesson, Barcelona (ES); Joan Farran, Barcelona (ES); Llorenc Rafecas, Barcelona (ES)

(73) Assignee: LABORATORIES DEL DR. ESTEVE, S.A., Barcelona (ES)

(21) Appl. No.: 12/919,855

(22) PCT Filed: Mar. 6, 2009
(86) PCT No.: PCT/EP2009/001640

§ 371 Date: Dec. 13, 2010

(30) Foreign Application Priority Data
Mar. 7, 2008 (EP) 08384002.5

(51) Int. Cl. A61K 31/616; C07C 59/64; C07C 69/017; C07C 65/05; C07C 57/30; C07C 229/42; C07C 51/41; C07C 67/00; C07C 227/16; A61K 31/192; A61K 31/196; A61P 29/00

(52) U.S. Cl. 514/165; 562/466; 560/143; 562/474; 562/496; 562/457; 562/492; 514/569; 514/159; 514/568; 514/570; 514/567

(57) ABSTRACT

The present invention relates to salts of Memantine and COX-INHIBITORs, their crystal form, the processes for preparation of the same and their uses for the treatment of various disorders, including pain.

The present invention relates to salts of Memantine and COX-INHIBITORs, their crystal form, and their specific polymorphs, the processes for preparation of the same and their uses as medicaments, more particularly for the treatment of pain.

Pain is a complex response that has been functionally categorized into sensory, autonomic, motor, and affective components. The sensory aspect includes information about stimulus location and intensity while the adaptive component may be considered to be the activation of endogenous pain modulation and motor planning for escape responses. The affective component appears to include evaluation of pain unpleasantness and stimulus threat as well as negative emotions triggered by memory and context of the painful stimulus.

In general, pain conditions can be divided into chronic and acute. Chronic pain includes neuropathic pain and chronic inflammatory pain, for example arthritis, or pain of unknown origin, as fibromyalgia. Acute pain usually follows non-neural tissue injury, for example tissue damage from surgery or inflammation, or migraine.

One compound whose main focus currently is not in pain, but which nevertheless did show initial success in diabetic neuropathy and also chronic and arthritic pain in animal models is the NMDA receptor antagonist Memantine. Besides these animal models a number of clinical trials were undertaken, which resulted also in proving the efficacy of Memantine in pain, but at least in one case did not reach the endpoint which was envisioned. Therefore, even though there is no doubt that Memantine has potential and efficacy in the treatment of pain, the current free base or hydrochloride salt used in the trials under certain less than optimal conditions did not seem to be sufficient for a clinical success. Therefore, there is a clear need for alternatives, especially new salts of Memantine that would enhance usability or efficacy of Memantine in pain, especially in neuropathic pain and under clinical conditions.

Memantine is currently marketed for the treatment of Alzheimer's disease. Memantine (1-amino-3,5-dimethyl-adamantane). Memantine—whose empirical formula as a free base is C₁₂H₂₁N—has a pKa of 10.7. Memantine free base has the following formula:

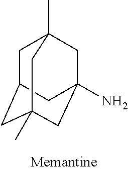

Memantine is available as a free base but also is available or described in form of a number of salts, including salts with HCl, HBr, HI, butenedioic acid, as nitrate, sulfate, phosphate, oxalate, citrate, methanesulfonate, toluenesulfonate, tartrate 1,6-hexandioate, 3-amino-propanesulfonate, N-vinylsuccinamic acid, or crotonic acid.

Nevertheless despite this, one of the main disadvantages of Memantine is its low solubility limiting its use in pharmaceutical formulations. Even though maybe partly overcome by use of the salts of Memantine described above, the big majority of them is either not very useful or difficult to formulate, has physiological drawbacks or is only available in very specific formulations. In addition the acidic partners of the Memantine in the salt are of no pharmaceutical value in themselves only adding—in some cases considerable—molecular weight to the active ingredient thus increasing the overall size of the pharmaceutical formulation without increasing the dosage. As in addition it is well-known that often there are a number of chemical difficulties to be overcome for obtaining salts of Memantine, there still is a clear need for salts of Memantine either
being active in pain or even more active when compared to Memantine base or hydrochloride salt; or
being easily obtainable, or
being easily crystallized, allowing more flexibility in formulating, or
being highly soluble, especially if compared to Memantine base, allowing better dissolution rates, especially if dissolving in an aqueous physiological surrounding, or
having as acidic partner of the Memantine a molecule having a beneficial pharmacological effect in itself, thus allowing for a highly efficient dose/weight relation of the active principle or
having a synergistic effect in the combination of Memantine and its acidic partner; or
allowing the use of a lower therapeutic dose of either Memantine and its acidic partner or of both.

Most desirably the salt should combine more than one, most preferably all of these advantages.

Besides Memantine there are a considerable number of drugs known to be useful in the treatment or management of pain. Thus, for example opioids are frequently used as analgesics in pain, obtaining the analgesic effect through their action on morphinic receptors, preferably the μ-receptors. Besides these derivatives of morphine, there are a number of other well-known analgesics in the market.

One well-known group of analgesic compounds are the well established COX-INHIBITORs which include the NSAIDs (Non steroidal anti-inflammatory drugs) and have analgesic activity in a number of pain symptoms, with Acetylsalicylic acid known under its trademark Aspirin—despite being more than 100 years old—being an outstandingly used pharmaceutical. Besides Aspirin other COX-INHIBITORS whose use generally is also centered on anti-inflammatory action like Ibuprofen, Naproxen or Diclofenac are among the worldwide most frequently applied pharmaceutical compounds. The basis of their activity is inhibition of cyclooxygenase (COX), one of the two activities of prostaglandine endoperoxide synthase (PGHS). It is a key enzyme in the prostaglandin pathway. For a number of COX-INHIBITORS the same problem as known for Memantine base, a low solubility in water exists. As an example, this is especially true for the very popular and widely used and distributed members of the group of COX-INHIBITORS, Naproxen, Diclofenac and Ibuprofen, whose poor solubility is a published fact, that has lead to considerably efforts for improvement by using solution enhancers etc. in their formulation. Accordingly the COX-INHIBITORS like Naproxen, dixclofenac or Ibuprofen hardly seemed to be partners of choice for improving solubility of another also nearly insoluble compound.

Nevertheless, to its surprise the applicant has now found that Memantine and COX-INHIBITORS having a carboxylic group can be combined to form a well-soluble mixed-salt.

Thus the object of the present invention is a salt of Memantine with a COX-INHIBITOR, wherein the COX-INHIBITOR has a carboxylic group.

These mixed salts are not only easily formed and crystallized they also considerable improve the solubility of Memantine, but often also of its COX-INHIBITOR-partner. Also this association of the two active principles into the same salt exhibits several further advantages. Being linked as ion and counter-ion, they behave as a single chemical entity, thus facilitating the treatments, formulation, dosage etc. In addition to that, with both Memantine and the COX-INHIBITOR being active analgesics these mixed salts are highly useful in the treatment of pain, especially also not losing any activity/weight by the addition of pharmacologically useless counterions. In addition the two active principles are complementing each other in the treatment especially of pain, but possibly also of various other diseases or symptoms. Thus, the mixed salts according to the invention do combine a high number of advantages over the state of the art.

The Applicant has further demonstrated the possibility to crystallize said salts. Even though also amorphous salts are also an aspect of the current invention, most preferred are crystalline salts. By that way the physico-chemical properties are improved. The formulation of the mixed salt is even easier with a solid to manipulate and an enhanced stability. The solubility, in particular the solubility of the Memantine—but also in some cases like Naproxen also of the COX-INHIBITOR salt—is also greatly augmented.

Another advantage is that the association of the two active principles into one unique species seems to allow for a better Pharmacokinetic/Pharmacodynamic (PKPD) including also a better penetration of the blood-brain barrier, which helps in the treatment of pain.

In general in most embodiments in which the salts of Memantine are used (e.g. for the treatment of pain, etc.) these salts would be formulated into a convenient pharmaceutical formulation or a medicament. Accordingly a desirable advantage of a Memantine salt, especially if crystallized, would show improved pharmaceutical properties and features, especially when compared to the free base or Memantine hydrochloride. Thus, the Memantine salt according to the invention should desirably show at least one, preferably more, of the following features:
to have a very small particle size, e.g. from 300 μm or lower; or
to be and/or remain essentially free of agglomerates; or
to be less or not very hygroscopic; or
to allow by selection of the counter-ion of the Memantine to help in formulating controlled release or immediate release formulations; or
to have a high chemical stability; or
   if given to a patient
to decrease the inter- and intra-subject variability in blood levels; or
to show a good absorption rate (e.g. increases in plasma levels or AUC); or
to show a high maximum plasma concentration (e.g. Cₘₐₓ); or
to show decreased time to peak drug concentrations in plasma (tₘₐₓ); or
to show changes in half life of the compound (t_{1/2}), in whichever direction this change is preferably directed.

Also, the Memantine salt according to the invention, should desirably show at least one, preferably more, of the following features
being active in pain or even more active when compared to Memantine free base or hydrochloride salt or to the COX INHIBITOR; or
being easily obtainable, or
being easily crystallized, allowing more flexibility in formulating, or
being highly soluble allowing good dissolution rates, especially if dissolving in an aqueous physiological surrounding, or
having as acidic partner of the Memantine a molecule having a beneficial pharmacological effect in itself, thus allowing for a highly efficient dose/weight relation of the active principle.

In one embodiment of the salt according to the invention the COX-INHIBITOR is selected from:
Acetylsalicylic Acid;
Triflusal;
HTB (2-hydroxy-4-trifluoromethyl benzoic acid);
Diflunisal;
Meclofenamic acid;
Mefenamic acid;
Niflumic acid;
Flufenamic acid.
Diclofenac;
Lonazolac;
Acemetacin;
Indomethacin;
Tolmetin;
Sulindac
Etodolac;
Keterolac
Flurbiprofen;
(RS)-Flurbiprofen;
Esflurbiprofen;
Ibuprofen;
(RS)-Ibuprofen;
S-(+)-Ibuprofen;
Ketoprofen;
(rac)-Ketoprofen
R-(−)-Ketoprofen
Bermoprofen;
Pelubiprofen;
Tenosal;
Aceneuramic acid;
Pirazolac;
Xinoprofen;
Flobufen;
Anirolac;
Zoliprofen;
Bromfenac;
Pemedolac;
Dexpemedolac;
Bindarit;
Romazarit;
Naproxen;
(S)-Naproxen;
Tiaprofenic acid;
Ketorolac;
Fenbufen;
Fenoprofen;
Flobufen; or
Oxaprozin.

In another embodiment of the salt according to the invention the COX-INHIBITOR is selected from:
Acetylsalicylic Acid;
Triflusal;
HTB (2-hydroxy-4-trifluoromethyl benzoic acid);
Diflunisal;
Meclofenamic acid;
Mefenamic acid;
Niflumic acid;
Flufenamic acid.
Diclofenac;
Lonazolac;
Acemetacin;
Indomethacin;
Tolmetin;
Sulindac
Etodolac;
Keterolac
Flurbiprofen;
(RS)-Flurbiprofen;
Esflurbiprofen;
(R)-Flurbiprofen;
Ibuprofen;
(RS)-Ibuprofen;
S-(+)-Ibuprofen;
R-(−)-Ibuprofen;
Ketoprofen;
(rac)-Ketoprofen
R-(−)-Ketoprofen
S-(+)-Ketoprofen
Bermoprofen;
Pelubiprofen;
Tenosal;
Aceneuramic acid;
Pirazolac;
Xinoprofen;
Flobufen;
Anirolac;
Zoliprofen;
Bromfenac;
Pemedolac;
Dexpemedolac;
Bindarit;
Romazarit;
Naproxen;
(S)-Naproxen;
(R)-Naproxen;
Tiaprofenic acid;
Ketorolac;
Fenbufen;
Fenoprofen;
Flobufen; or
Oxaprozin.

All of these COX-INHIBITORS are well-known and/or widely marketed drugs. In general all of these COX-Inhibitors which have at least one stereogenic center are to be understood as being included herein in their racemic form or as diastereoisomers or enantiomers or mixtures thereof.

In another embodiment of the salt according to the invention the COX-INHIBITOR is selected from:
Salicylates,
Anthranilates,
Arylacetic acids/Arylalkanoic acids,
Arylpropionic acids.

In another embodiment of the salt according to the invention the Salicylates are selected from:
Acetylsalicylic acid;
Triflusal;
HTB (2-hydroxy-4-trifluoromethyl benzoic acid); or
Diflunisal;
preferably are
Acetylsalicylic acid;
HTB (2-hydroxy-4-trifluoromethyl benzoic acid); or
Triflusal.

In another embodiment of the salt according to the invention the Anthranilates are selected from:
Meclofenamic acid;
Mefenamic acid;
Niflumic acid; or
Flufenamic acid.

In another embodiment of the salt according to the invention the Arylacetic Acids/Arylalkanoic Acids are selected from:
Diclofenac;
Lonazolac;
Acemetacin;
Indomethacin;
Tolmetin; or
Sulindac
Etodolac;
Keterolac;
preferably from
Diclofenac;
Lonazolac;
Acemetacin;
Indomethacin;
Tolmetin; or
Sulindac;
most preferably is
Diclofenac.

In another embodiment of the salt according to the invention the Arylpropionic acids are selected from:
Flurbiprofen;
(RS)-Flurbiprofen;
Esflurbiprofen;
Ibuprofen;
(RS)-Ibuprofen;
S-(+)-Ibuprofen;
Ketoprofen;
(rac)-Ketoprofen;
R-(−)-Ketoprofen;
Naproxen;
(S)-Naproxen;
Tiaprofenic acid;
Ketorolac;
Fenbufen;
Fenoprofen;
Flobufen;
Oxaprozin;
Tolmetin;
Xinoprofen;
Flobufen;
Zoliprofen;
Bermoprofen; or
Pelubiprofen;
preferably from
Flurbiprofen;
(RS)-Flurbiprofen;
Esflurbiprofen;
Ibuprofen;
(RS)-Ibuprofen;
S-(+)-Ibuprofen;
Ketoprofen;
(rac)-Ketoprofen;
R-(−)-Ketoprofen;
Naproxen;
(S)-Naproxen;
Tiaprofenic acid; or
Ketorolac;
preferably is
(RS)-Flurbiprofen;
Esflurbiprofen;
(RS)-Ibuprofen;
S-(+)-Ibuprofen;
(rac)-Ketoprofen;
R-(−)-Ketoprofen; or
(S)-Naproxen.

In a further embodiment of the salt according to the invention the Arylpropionic Acids are selected from:
Flurbiprofen;
(RS)-Flurbiprofen;
Esflurbiprofen;
(R)-Flurbiprofen;
Ibuprofen;
(RS)-Ibuprofen;
S-(+)-Ibuprofen;
R-(−)-Ibuprofen;
Ketoprofen;
(rac)-Ketoprofen;
R-(−)-Ketoprofen;
S-(+)-Ketoprofen;
Naproxen;
(S)-Naproxen;
(R)-Naproxen;
Tiaprofenic acid;
Ketorolac;
Fenbufen;
Fenoprofen;
Flobufen;
Oxaprozin;
Tolmetin;
Xinoprofen;
Flobufen;
Zoliprofen;
Bermoprofen; or
Pelubiprofen;
preferably from
Flurbiprofen;
(RS)-Flurbiprofen;
Esflurbiprofen;
Ibuprofen;
(RS)-Ibuprofen;
S-(+)-Ibuprofen;
Ketoprofen;
(rac)-Ketoprofen;
R-(−)-Ketoprofen;
Naproxen;
(S)-Naproxen;
Tiaprofenic acid; or
Ketorolac;
preferably is
(RS)-Flurbiprofen;
Esflurbiprofen;
(RS)-Ibuprofen;
S-(+)-Ibuprofen;
(rac)-Ketoprofen;
R-(−)-Ketoprofen; or
(S)-Naproxen.

Another embodiment is a salt of Memantine with a COX-INHIBITOR according to the invention selected from Memantine-Ibuprofen salt, Memantine-Flurbiprofen salt, Memantine-Diclofenac salt, Memantine-Acetylsalicylic acid salt, Memantine-(S)-Naproxen salt, Memantine/Triflusal salt, or Memantine-2-hydroxy-4-trifluoromethyl benzoic acid (HTB) salt.

Another embodiment of the invention is a Memantine-Ibuprofen salt.

Another embodiment of the invention is a Memantine-Diclofenac salt.

Another embodiment of the invention is a Memantine-Acetylsalicylic Acid salt.

Another embodiment of the invention is a Memantine-(S)-Naproxen salt.

Another embodiment of the invention is a Memantine-Flurbiprofen salt.

Another embodiment of the invention is a Memantine-Triflusal salt.

Another embodiment of the invention is a Memantine-HTB salt.

As the applicant has shown the possibility to crystallize said salts according to the invention a crystalline form of a salt according to the invention it is a separate, highly interesting aspect of the current invention.

Another embodiment the present invention relates to a process for the production of a salt according to the invention as described above comprising the steps of:
dissolving a COX-INHIBITOR with a carboxylic group either as a free acid or as a salt together with, or after, or before, Memantine either as a free base or as a salt in an organic solvent,
stirring the mixture obtained at a temperature between 0° C. and 80° C.,
evaporating the solvent and/or evaporating the solvent, and
drying of the resulting product.

Preferably in the process above
the organic solvent is selected from acetone, acetonitrile, isobutyl acetate, heptane, methanol, tetrahydrofuran, isopropanol, ethanol or cyclohexane; and/or
the solvent is evaporated under high vacuum; and/or
the ratio of Memantine to COX-INHIBITOR is 1:1 to 1:2, preferably is 1:1; and/or
the Memantine dissolved is a free base.

Both parts of the salt are well-known drugs sometimes used for a long time worldwide. Due to the therapeutic interest in Memantine in the treatment of pain symptoms like diabetic neuropathy and the well-known properties of COX-INHIBITORS in this field of medical indication, a further object of the present invention is a medicament containing a Memantine-COX-INHIBITOR salt, or its crystalline form according to the invention.

Thus the invention also concerns a medicament comprising at least one salt according to the invention as described above (or in preferred aspects as will be described below) and optionally one or more pharmaceutically acceptable excipients.

A further object of the invention is a pharmaceutical composition characterized in that it comprises an efficient amount of at least one salt according to the invention as described above (or in preferred aspects as will be described below) or its crystalline form, in a physiologically acceptable medium.

The medicament according to the present invention may be in any form suitable for the application to humans and/or animals, preferably humans including infants, children and adults and can be produced by standard procedures known to those skilled in the art. The medicament of the present invention may for example be administered parentally, including intramuscular, intraperitoneal, or intravenous injection; or orally, including administration as tablets, pellets, granules, capsules, lozenges, aqueous or oily solutions, suspensions, emulsions, sprays or as reconstituted dry powdered form with a liquid medium.

Typically, the medicaments according to the present invention may contain 1-60% by weight of one or more of the salts or their crystalline form as defined herein and 40-99% by weight of one or more auxiliary substances (additives/excipients).

The compositions of the present invention may also be administered topically or via a suppository.

The daily dosage for humans and animals may vary depending on factors that have their basis in the respective species or other factors, such as age, sex, weight or degree of illness and so forth. The daily dosage for humans preferably is in the range of 10 to 2000 milligrams of active substance to be administered during one or several intakes per day.

A further aspect of the invention relates to the use of a salt according to the invention as described above (or in preferred aspects as will be described below) for the treatment of pain, preferably acute pain, chronic pain, neuropathic pain, hyperalgesia, allodynia or cancer pain, including diabetic neuropathy or osteoarthritis. Preferably this use is provided for in form of a medicament or a pharmaceutical composition according to the invention as described above.

Another object of the current invention is a method of treatment of pain, preferably acute pain, chronic pain, neuropathic pain, hyperalgesia, allodynia or cancer pain, including diabetic neuropathy or osteoarthritis, by providing to a patient in need thereof a sufficient amount of a salt according to the invention as described above (or in preferred aspects as will be described below). Preferably a salt according to the invention or its crystalline form according to the invention is provided in physiologically suitable form like e.g. in form of a medicament or a pharmaceutical composition according to the invention as described above.

An interesting COX-INHIBITOR to be combined with Memantine is the marketed drug Naproxen, whose chemical name is 2(S)-(6-methoxy-2-naphthyl)propionic acid, and which is also described as a physiologically acceptable salt. It has an empirical formula of C₁₄H₁₄O₃, a Mp of 153° C. and a pKa of 4.2.

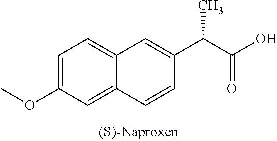

Thus, another very preferred aspect of the invention relates to a Memantine-(S)-Naproxen salt.

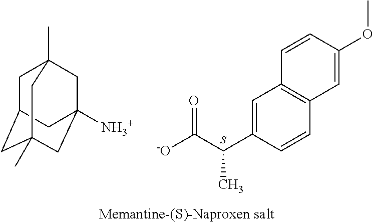

A second object of this preferred aspect of the invention is a crystalline form of a Memantine-(S)-Naproxen salt.

More particularly, the invention concerns a Memantine-(S)-Naproxene salt or a crystalline form of Memantine-(S)-Naproxene salt, characterized in that it shows a Fourier Transform Infra Red pattern with absorption bands at 2947, 2906, 2864, 2848, 2648, 2555, 2195, 1633, 1604, 1553, 1536, 1378, 1361, 1346, 1247, 1211, 1036, 858, 814, and 693 cm⁻¹.

The invention also concerns a Memantine-(S)-Naproxene salt or a crystalline form of Memantine-(S)-Naproxene salt, showing a powder X-ray diffraction pattern (XRPD) with peaks [2θ] at 6.1, 7.8, 8.1, 11.3, 12.0, 14.0, 14.7, 15.7, 16.2, 17.5, 18.1, 18.4, 19.2, 19.6, 20.0, 22.1, 22.5, 23.5, and 24.4 (°) (see also FIG. 3; the 2θ values refer to those obtained using copper radiation (Cu_{Kα1} 1.54060 Å)).

The invention also concerns a Memantine-(S)-Naproxene salt or a crystalline form of Memantine-(S)-Naproxene salt showing an X-ray powder diffraction spectrum with peaks expressed in d-Value in Å at 14.58, 11.38, 10.88, 7.84, 7.36, 6.34, 6.03, 5.64, 5.47, 5.08, 4.89, 4.82, 4.61, 4.52, 4.45, 4.03, 3.96, 3.79, and 3.65.

The invention also encompasses a Memantine-(S)-Naproxene salt or a crystalline form of Memantine-(S)-Naproxen salt with a ¹H NMR spectrum as described in Example 1 in D4-methanol at 400 MHz.

In another embodiment, the present invention concerns a crystalline form of Memantine-(S)-Naproxen salt, characterized in that it crystallizes in the monoclinic system with the following unit cell dimensions:
a=24.34 Å
b=6.65 Å
c=15.63 Å
β angle of 109.18°

The single crystal structure is shown in FIG. 4).

The crystalline form of Memantine-(S)-Naproxen salt according to the present invention is characterized in that the endothermic sharp peak corresponding to the melting point has an onset at 173° C., measured by DSC analysis (10° C./min) (see FIG. 1).

The TG analysis of the crystalline form according to the invention shows no weight loss at temperatures lower than the melting point (see FIG. 2).

Another interesting COX-INHIBITOR to be combined with Memantine for the use according to the invention is the marketed drug Triflusal (2-acetoxy-4-trifluoromethyl-benzoic acid). Triflusal—having an empirical formula of C₁₀H₇F₃O₄ has a Mp of 116° C. and a pKa of 3.34—. has the following formula:

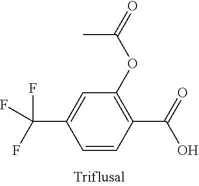

Thus, a very preferred aspect of the invention relates to a Memantine-Triflusal salt or a salt of Memantine with HTB (2-hydroxy-4-trifluoromethyl benzoic acid), a metabolite of Triflusal.

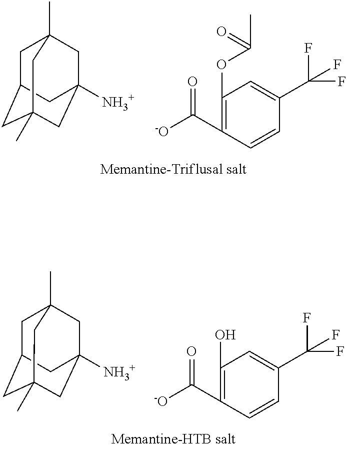

The Applicant has further demonstrated the possibility to crystallize said salts. By that way the physico-chemical properties are improved. The formulation of the mixed salt is even easier with a solid to manipulate and an enhanced stability. The solubility, in particular the solubility of the Memantine is also greatly augmented.

A further object of this invention is a crystalline form of a Memantine-Triflusal salt.

More particularly, the invention concerns a Memantine-Triflusal salt or a crystalline form of Memantine-Triflusal salt, characterized in that it shows a Fourier Transform Infra Red pattern with absorption bands at 2947, 2908, 2867, 1777, 1629, 1587, 1560, 1411, 1385, 1366, 1333, 1207, 1196, 1170, 1159, 1128, 1108, 1065, 944, and 897 cm⁻¹.

The invention also concerns a Memantine-Triflusal salt or a crystalline form of Memantine-Triflusal salt, showing a powder X-ray diffraction pattern (XRPD) with peaks [2θ] at 7.3, 10.0, 11.4, 11.7, 12.5, 14.5, 15.0, 15.4, 15.9, 16.2, 16.9, 17.8, 18.1, 18.7, 19.5, 19.9, 20.8, 21.1, 22.0, 22.9, 23.4, 25.1, 26.5, 27.3, and 28.9 (°) (see also FIG. 7; the 2θ values refer to those obtained using copper radiation (Cu_{Kα1} 1.54060 Å)).

The invention also concerns a Memantine-Triflusal salt or a crystalline form of Memantine-Triflusal salt showing an X-ray powder diffraction spectrum with peaks expressed in d-Value in Å at 12.11, 8.88, 7.76, 7.55, 7.10, 6.10, 5.93, 5.76, 5.56, 5.46, 5.25, 5.00, 4.90, 4.74, 4.55, 4.47, 4.27, 4.22, 4.05, 3.89, 3.80, 3.55, 3.37, 3.27, and 3.09.

The invention also encompasses a Memantine-Triflusal salt or a crystalline form of Memantine-Triflusal salt with a ¹H NMR spectrum according to Example 2 in D4-chloroform at 400 MHz.

In another embodiment, the present invention concerns a crystalline form of Memantine-Triflusal salt, characterized in that it crystallizes in the monoclinic system with the following unit cell dimensions:
a=30.96 Å
b=13.62 Å
c=11.94 Å
β angle of 112.34°

The single crystal structure is shown in FIG. 8).

The crystalline form of Memantine-Triflusal salt according to the present invention is characterized in that the endothermic sharp peak corresponding to the melting point has an onset at 133° C., measured by DSC analysis (10° C./min), see FIG. 5.

Another further aspect of the invention is a crystalline form of a Memantine-HTB salt.

More particularly, the invention concerns a Memantine-HTB salt or a crystalline form of Memantine/HTB salt, characterized in that it shows a Fourier Transform Infra Red pattern with absorption bands at 2949, 2919, 2849, 1668, 1593, 1501, 1454, 1438, 1389, 1354, 1336, 1256, 1240, 1175, 1152, 1122, 1062, 921, 872, 846, 826, 797, 750, 703, 578 and 490 cm⁻¹.

The invention also concerns a Memantine-HTB salt or a crystalline form of Memantine/HTB salt, showing a powder X-ray diffraction pattern (XRPD) with peaks [2θ] at 6.7, 8.6, 10.2, 11.4, 13.3, 14.1, 15.0, 15.5, 16.6, 17.1, 17.6, 17.9, 18.3, 19.0, 19.8, 20.8, 22.1, 22.5, 22.8, 24.5, 25.3, 25.8, 26.7, 27.2, 29.3, 32.8, and 39.5 (°) (see also FIG. 11; the 2θ values refer to those obtained using copper radiation (Cu_{Kα1} 1.54060 Å)).

The invention also concerns a Memantine-HTB salt or a crystalline form of Memantine/HTB salt showing an X-ray powder diffraction spectrum with peaks expressed in d-Value in Å at 13.18, 10.32, 8.65, 7.77, 6.65, 6.26, 5.91, 5.72, 5.35, 5.20, 5.04, 4.96, 4.84, 4.68, 4.48, 4.27, 4.02, 3.94, 3.91, 3.63, 3.52, 3.46, 3.34, 3.28, 3.05, 2.73, and 2.28.

The invention also encompasses a Memantine-HTB salt or a crystalline form of Memantine/HTB salt with a ¹H NMR spectrum of Example 3 in D4-chloroform at 400 MHz.

In another embodiment, the present invention concerns a crystalline form of Memantine-HTB salt, characterized in that it crystallizes in the triclinic system with the following unit cell dimensions:
a=7.13 Å
b=11.09 Å
c=13.55 Å
α angle of 94.45°
β angle of 94.77°
γ angle of 108.37°.

The single crystal structure is shown in FIG. 12).

The crystalline form of Memantine-HTB salt according to the present invention is characterized in that the endothermic sharp peak corresponding to the melting point has an onset at 206° C., measured by DSC analysis (10° C./min) (see FIG. 9).

Another interesting COX-INHIBITOR to be combined with Memantine for the use according to the invention is the marketed drug Ibuprofen (2-[4-(2-methylpropyl)phenyl]propanoic acid), especially (S)-Ibuprofen. Ibuprofen—having an empirical formula of C₁₃H₁₈O₂ has a Mp of 76° C. and a pKa 4.4—has the following formula:

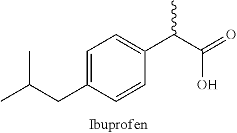

Thus, a very preferred aspect of the invention relates to a Memantine-Ibuprofen salt or a Memantine-(S)-Ibuprofen salt.

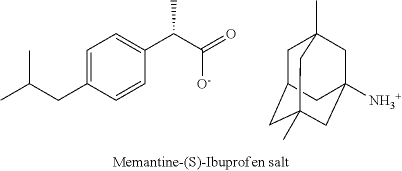

The Applicant has further demonstrated the possibility to crystallize said salts. By that way the physico-chemical properties are improved.

A further object of this invention is a Memantine-Ibuprofen salt, a Memantine-(S)-Ibuprofen salt or a crystalline form of a Memantine-Ibuprofen salt or of a Memantine-(S)-Ibuprofen salt.

A further embodiment of this invention is a Memantine-(S)-Ibuprofen salt or a crystalline form of a Memantine-(S)-Ibuprofen salt showing a Fourier Transform Infra Red pattern with absorption bands at 2954, 2649, 2213, 1638, 1548, 1454, 1380, 1361, 1282, 1060, 876, 799, 726 and 547 cm⁻¹.

The invention also concerns a Memantine-(S)-Ibuprofen salt or a crystalline form of a Memantine-(S)-Ibuprofen salt showing a powder X-ray diffraction pattern (XRPD) with peaks [2θ] at 6.6, 9.2, 10.4, 14.3, 14.6, 15.0, 16.5, 16.8, 17.1, 18.5, 18.9, 19.1, 19.8, 20.0, 20.9, 21.6, 23.4, 25.0, 27.1, 27.9, 28.8, 29.2, 29.9, 31.8, 34.5, and 36.7 (°) (see FIG. 15; the 2θ values refer to those obtained using copper radiation (Cu_{Kα1} 1.54060 Å)).

The invention also concerns a Memantine-(S)-Ibuprofen salt or a crystalline form of a Memantine-(S)-Ibuprofen salt showing an X-ray powder diffraction spectrum with peaks expressed in d-Value in Å at 13.43, 9.58, 8.48, 6.19, 6.05, 5.90, 5.39, 5.29, 5.20, 4.80, 4.70, 4.64, 4.49, 4.45, 4.25, 4.12, 3.80, 3.56, 3.29, 3.20, 3.10, 3.06, 2.99, 2.81, 2.56, and 2.45.

The invention also encompasses a Memantine-(S)-Ibuprofen salt or a crystalline form of Memantine-(S)-Ibuprofen salt with a ¹H NMR spectrum of Example 4 in D4-chloroform at 400 MHz.

A further embodiment of this invention is a Memantine-(S)-Ibuprofen salt or a crystalline form of a Memantine-(S)-Ibuprofen salt crystallizing in the orthorhombic system with the following unit cell dimensions:
a=6.57 Å
b=18.96 Å
c=19.20 Å

The single crystal structure is shown in FIG. 16).

A further embodiment of this invention is a crystalline form of a Memantine-(S)-Ibuprofen salt wherein the endothermic sharp peak corresponding to the melting point has an onset at 116° C. (see FIG. 13).

Another interesting COX-INHIBITOR to be combined with Memantine for the use according to the invention is the marketed drug Diclofenac (2-(2-(2,6-dichlorophenylamino)phenyl)acetic acid). Diclofenac—having an empirical formula of C₁₄H₁₁Cl₂NO₂ has a Mp of 174° C. and a pka 4.0—has the following formula:

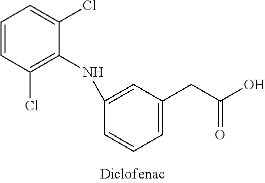

Thus, a very preferred aspect of the invention relates to a Memantine-Diclofenac salt.

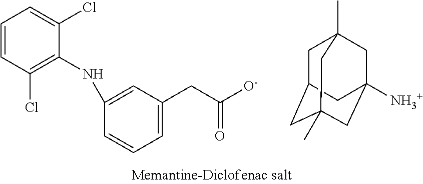

The Applicant has further demonstrated the possibility to crystallize said salts. By that way the physico-chemical properties are improved.

A further object of this invention is a crystalline form of a Memantine-Diclofenac salt.

A further embodiment of this invention is a Memantine-Diclofenac salt or a crystalline form of a Memantine-Diclofenac salt showing a Fourier Transform Infra Red pattern with absorption bands at 3212, 2946, 2848, 2707, 2654, 1633, 1548, 1504, 1495, 1467, 1452, 1386, 873, 767, 745 and 718 cm⁻¹.

The invention also concerns a Memantine-Diclofenac salt or a crystalline form of a Memantine-Diclofenac salt showing a powder X-ray diffraction pattern (XRPD) with peaks [2θ] at 8.2, 10.6, 12.4, 14.0, 14.5, 16.7, 17.9, 18.6, 19.4, 21.0, 21.9, 23.9, 24.7, 25.6, 27.7, 31.4, and 38.4 [2θ] (°) (see also FIG. 19; the 2θ values refer to those obtained using copper radiation (Cu_{Kα1} 1.54060 Å)).

The invention also concerns a Memantine-Diclofenac salt or a crystalline form of a Memantine-Diclofenac salt showing an X-ray powder diffraction spectrum with peaks expressed in d-Value in Å at 10.82, 8.33, 7.14, 6.31, 6.10, 5.30, 4.94, 4.77, 4.57, 4.23, 4.06, 3.72, 3.61, 3.48, 3.22, 2.85, and 2.34.

The invention also encompasses a Memantine-Diclofenac salt or a crystalline form of Memantine-Diclofenac salt with a ¹H NMR spectrum of Example 5 in D4-chloroform at 400 MHz.

A further embodiment of this invention is a crystalline form of a Memantine-Diclofenac salt crystallizing in the monoclinic system with the following unit cell dimensions:
a=16.94 Å
b=6.78 Å
c=22.04 Å
β angle of 98.51°

The single crystal structure is shown in FIG. 20).

A further embodiment of this invention is a Memantine-Diclofenac salt or a crystalline form of a Memantine-Diclofenac salt wherein the endothermic sharp peak corresponding to the melting point has an onset at 207° C. (see FIG. 17).

Another interesting COX-INHIBITOR to be combined with Memantine for the use according to the invention is the marketed drug Acetylsalicylic acid, widely known under its trademark aspirin. Acetylsalicylic acid—having an empirical formula of C₉H₈O₄ and a Mp of 135° C. and a pKa 3.5—has the following formula:

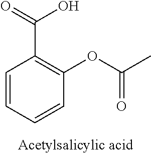

Thus, a very preferred aspect of the invention relates to a Memantine-Acetylsalicylic acid salt.

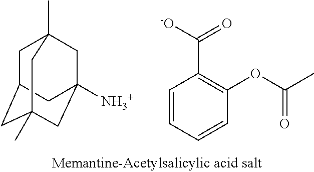

The Applicant has further demonstrated the possibility to crystallize said salts. By that way the physico-chemical properties are improved.

A further object of this invention is a crystalline form of a Memantine-Acetylsalicylic acid salt.

A further embodiment of this invention is a Memantine-Acetylsalicylic acid salt or a crystalline form of a Memantine-Acetylsalicylic acid salt showing a Fourier Transform Infra Red pattern with absorption bands at 2910, 2638, 1766, 1752, 1623, 1606, 1590, 1552, 1386, 1367, 1219, 1196, 1091, 918 and 750 cm⁻¹.

The invention also concerns a Memantine-Acetylsalicylic acid salt or a crystalline form of a Memantine-Acetylsalicylic acid salt showing a powder X-ray diffraction pattern (XRPD) with peaks [2θ] at 7.1, 7.4, 8.6, 11.6, 12.4, 12.8, 13.2, 14.2, 15.9, 16.4, 16.8, 17.4, 18.3, 18.5, 18.8, 19.7, 20.2, 22.0, 22.6, 23.3, 24.2, 24.7, 25.7, 26.7, and 27.8 (°) (see also FIG. 23; the 2θ values refer to those obtained using copper radiation (Cu_{Kα1} 1.54060 Å)).

The invention also concerns a Memantine-Acetylsalicylic acid salt or a crystalline form of a Memantine-Acetylsalicylic acid salt showing an X-ray powder diffraction spectrum with peaks expressed in d-Value in Å at 12.44, 12.02, 10.25, 7.60, 7.12, 6.92, 6.69, 6.24, 5.56, 5.42, 5.28, 5.10, 4.84, 4.79, 4.71, 4.51, 4.40, 4.05, 3.93, 3.83, 3.68, 3.61, 3.46, 3.34, and 3.21.

The invention also encompasses a Memantine-Acetylsalicylic acid salt or a crystalline form of a Memantine-Acetylsalicylic acid salt with a ¹H NMR spectrum of Example 6 in D4-chloroform at 400 MHz.

A further embodiment of this invention is a crystalline form of a Memantine-Acetylsalicylic acid salt crystallizing in the triclinic system with the following unit cell dimensions:
a=11.57 Å
b=14.50 Å
c=14.52 Å
α angle of 115.68°
β angle of 105.59°
γ angle of 101.75°

The single crystal structure is shown in FIG. 24).

A further embodiment of this invention is a Memantine-Acetylsalicylic acid salt or a crystalline form of a Memantine-Acetylsalicylic acid salt wherein the endothermic sharp peak corresponding to the melting point has an onset at 127° C. (see FIG. 21).

Another interesting COX-INHIBITOR to be combined with Memantine for the use according to the invention is the marketed drug Flurbiprofen (2-(3-fluoro-4-phenyl-phenyl)propanoic acid). Flurbiprofen is marketed as racemate and its (R)-enantiomer is in clinical development. Flurbiprofen—having an empirical formula of C₁₅H₁₃FO₂ and a Mp of 117° C. and a pKa 4.2—has the following formula:

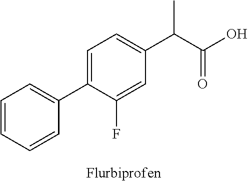

Thus, a very preferred aspect of the invention relates to a Memantine-Flurbiprofen salt.

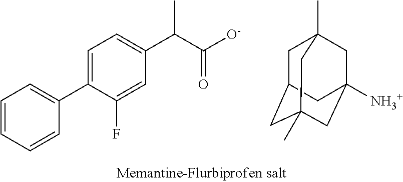

The Applicant has further demonstrated the possibility to crystallize said salts. By that way the physico-chemical properties are improved.

A further object of this invention is a crystalline form of a Memantine-Flurbiprofen salt.

A further embodiment of this invention is a crystalline form of a Memantine-Flurbiprofen salt which crystallizes as Memantine-(R)-Flurbiprofen (1:1).

A further embodiment of this invention is a crystalline form of a Memantine-Flurbiprofen salt which crystallizes as (rac)-Memantine-Flurbiprofen (1:1) or racemate of Memantine-Flurbiprofen (1:1) (Memantine-(R)-Flurbiprofen (1:1) and Memantine-(S)-Flurbiprofen (1:1))

Hereinafter (rac) signifies racemate or racemic mixture.

A further embodiment of this invention is a Memantine-Flurbiprofen salt or a crystalline form of a Memantine-Flurbiprofen salt crystallized as Memantine-(R)-Flurbiprofen (1:1) or racemate of Memantine-Flurbiprofen (1:1) in a crystalline Form (A) showing a Fourier Transform Infra Red pattern with absorption bands at 2948, 2903, 2842, 2647, 1636, 1552, 1483, 1455, 1417, 1379, 1359, 1316, 1263, 1131, 766, 726 and 698 cm⁻¹.

The invention also concerns a Memantine-Flurbiprofen salt or a crystalline form of a Memantine-Flurbiprofen salt crystallized as Memantine-(R)-Flurbiprofen (1:1) or racemate of Memantine-Flurbiprofen (1:1) in a crystalline Form (A) showing a powder X-ray diffraction pattern (XRPD) with peaks [2θ] at 6.6, 9.3, 10.4, 14.2, 14.7, 15.0, 16.4, 16.8, 17.0, 18.6, 18.8, 19.2, 19.8, 20.8, 21.5, 23.0, 23.3, 23.8, 24.9, 26.4, 27.0, 27.4, 28.0, 28.6, and 29.0(°) (see also FIG. 27; the 2θ values refer to those obtained using copper radiation (Cu_{Kα1} 1.54060 Å)).

The invention also concerns a Memantine-Flurbiprofen salt or a crystalline form of a Memantine-Flurbiprofen salt crystallized as Memantine-(R)-Flurbiprofen (1:1) or racemate of Memantine-Flurbiprofen (1:1) in a crystalline Form (A) showing an X-ray powder diffraction spectrum with peaks expressed in d-Value in Å at 13.40, 9.50, 8.50, 6.23, 6.03, 5.92, 5.42, 5.29, 5.21, 4.77, 4.71, 4.63, 4.50, 4.26, 4.13, 3.87, 3.82, 3.74, 3.58, 3.37, 3.30, 3.26, 3.18, 3.12, and 3.08.

The invention also encompasses a Memantine-Flurbiprofen salt or a crystalline form of a Memantine-Flurbiprofen salt crystallized as Memantine-(R)-Flurbiprofen (1:1) or racemate of Memantine-Flurbiprofen (1:1) in a crystalline Form (A) with a ¹H NMR spectrum of Example 7 in D4-chloroform at 400 MHz.

A further embodiment of this invention is a crystalline form of a Memantine-Flurbiprofen salt crystallized as Memantine-(R)-Flurbiprofen (1:1) or racemate of Memantine-Flurbiprofen (1:1) in a crystalline Form (A) which crystallizes in the orthorhombic system with the following unit cell dimensions:
a=6.61 Å
b=19.10 Å
c=19.12 Å

The single crystal structure is shown in FIG. 28).

A further embodiment of this invention is a Memantine-Flurbiprofen salt or a crystalline form of a Memantine-Flurbiprofen salt crystallized as Memantine-(R)-Flurbiprofen (1:1) or racemate of Memantine-Flurbiprofen (1:1) in a crystalline Form (A) showing an endothermic sharp peak corresponding to the melting point at an onset at 124° C. (see FIG. 25).

A further embodiment of this invention is a Memantine-Flurbiprofen salt or a crystalline form of a Memantine-Flurbiprofen salt crystallized as Memantine-(rac)-Flurbiprofen (1:1) in a crystalline Form (B) showing a Fourier Transform Infra Red pattern with absorption bands at 2949, 2916, 2846, 2646, 1635, 1557, 1483, 1455, 1417, 1377, 1358, 1319, 1264, 1130, 926, 766, 726 and 698 cm⁻¹.

The invention also concerns a Memantine-Flurbiprofen salt or a crystalline form of a Memantine-Flurbiprofen salt crystallized as Memantine-(rac)-Flurbiprofen (1:1) in a crystalline Form (B) showing a powder X-ray diffraction pattern (XRPD) with peaks [2θ] at 5.9, 7.9, 9.3, 11.9, 13.8, 14.5, 14.9, 15.6, 16.5, 17.2, 17.8, 18.7, 20.1, 22.1, 23.8, 24.9, 26.3, 28.0, and 29.3 (°) (see also FIG. 31; the 2θ values refer to those obtained using copper radiation (Cu_{Kα1} 1.54060 Å)).

The invention also concerns a Memantine-Flurbiprofen salt or a crystalline form of a Memantine-Flurbiprofen salt crystallized as Memantine-(rac)-Flurbiprofen (1:1) in a crystalline Form (B) showing an X-ray powder diffraction spectrum with peaks expressed in d-Value in Å at 14.87, 11.27, 9.53, 7.44, 6.42, 6.10, 5.97, 5.67, 5.37, 5.15, 4.98, 4.75, 4.42, 4.03, 3.74, 3.57, 3.39, 3.18, and 3.06.

The invention also encompasses a Memantine-Flurbiprofen salt or a crystalline form of a Memantine-Flurbiprofen salt crystallized as Memantine-(R)-Flurbiprofen (1:1) or Memantine-(rac)-Flurbiprofen (1:1) in a crystalline Form (B) with a ¹H NMR spectrum of Example 8 in D4-chloroform at 400 MHz.

A further embodiment of this invention is a Memantine-Flurbiprofen salt or a crystalline form of a Memantine-Flurbiprofen salt crystallized as Memantine-(rac)-Flurbiprofen (1:1) in a crystalline Form (B) showing an endothermic sharp peak corresponding to the melting point at an onset at 129° C. (see FIG. 29).

A further embodiment of this invention is a Memantine-Flurbiprofen salt or a crystalline form of a Memantine-Flurbiprofen salt crystallized as Memantine-(rac)-Flurbiprofen (1:1) in a crystalline Form (C) showing a Fourier Transform Infra Red pattern with absorption bands at 2916, 2637, 1625, 1553, 1483, 1456, 1416, 1380, 1355, 1128, 926, 874, 765 and 698 cm⁻¹.

The invention also concerns a Memantine-Flurbiprofen salt or a crystalline form of a Memantine-Flurbiprofen salt crystallized as Memantine-(rac)-Flurbiprofen (1:1) in a crystalline Form (C) showing a powder X-ray diffraction pattern (XRPD) with peaks [2θ] at 5.0, 7.4, 7.8, 9.0, 9.9, 10.5, 10.9, 12.5, 13.1, 13.7, 15.0, 15.4, 15.9, 16.8, 17.3, 17.8, 18.4, 19.0, 20.2, 20.7, 21.5, 22.3, 22.8, 23.5, 24.4, 26.3, 27.2, 28.2, and 30.0 (°) (see also FIG. 34; the 2θ values refer to those obtained using copper radiation (Cu_{Kα1} 1.54060 Å)).

The invention also concerns a Memantine-Flurbiprofen salt or a crystalline form of a Memantine-Flurbiprofen salt crystallized as Memantine-(rac)-Flurbiprofen (1:1) in a crystalline Form (C) showing an X-ray powder diffraction spectrum with peaks expressed in d-Value in Å at 17.52, 11.95, 11.29, 9.87, 8.90, 8,39, 8.08, 7.10, 6.74, 6.44, 5.91, 5.74, 5.58, 5.27, 5.12, 4.97, 4.82, 4.67, 4.39, 4.29, 4.14, 4.00, 3.91, 3.79, 3.65, 3.39, 3.28, 3.17, and 2.98.

The invention also encompasses a Memantine-Flurbiprofen salt or a crystalline form of a Memantine-Flurbiprofen salt crystallized as Memantine-(R)-Flurbiprofen (1:1) or Memantine-(rac)-Flurbiprofen (1:1) in a crystalline Form (C) with a ¹H NMR spectrum of Example 9 in D4-chloroform at 400 MHz.

A further embodiment of this invention is a Memantine-Flurbiprofen salt or a crystalline form of a Memantine-Flurbiprofen salt crystallized as Memantine-(rac)-Flurbiprofen (1:1) in a crystalline Form (C) showing an endothermic sharp peak corresponding to the melting point at an onset at 134° C. (see FIG. 32).

The present invention is illustrated below with the help of the following figures and examples. These illustrations are given solely by way of Example and do not limit the invention.

### BRIEF DESCRIPTION OF THE FIGURES

FIG. 1: DSC analysis of crystalline form of Memantine-(S)-Naproxen salt.

The DSC analysis of the crystalline form of Memantine-(S)-Naproxen salt is shown measured as described in Example 1.

FIG. 2: TG analysis of crystalline form of Memantine-(S)-Naproxen salt.

The TG analysis of the crystalline form of Memantine-(S)-Naproxen salt is shown measured as described in Example 1.

FIG. 3: Powder X-ray diffraction pattern of crystalline form of Memantine-(S)-Naproxen salt (XRPD).

The powder X-ray diffraction pattern of the crystalline form of Memantine-(S)-Naproxen salt is shown measured as described in Example 1.

FIG. 4: Crystal structure of Memantine-(S)-Naproxen salt

The crystal structure as determined from the single crystal X-ray diffraction is given.

FIG. 5: DSC analysis of crystalline form of Memantine-Triflusal salt

The DSC analysis of the crystalline form of the Memantine-Triflusal salt are shown measured as described in Example 2.

FIG. 6: TG analysis of crystalline form of Memantine-Triflusal salt

The TG analysis of the crystalline form of the Memantine-Triflusal salt are shown measured as described in Example 2.

FIG. 7: Powder X-ray diffraction pattern of crystalline form of Memantine-Triflusal salt (XRPD)

The powder X-ray diffraction pattern of the crystalline form of the Memantine-Triflusal salt is shown measured as described in Example 2.

FIG. 8: Crystal structure of Memantine-Triflusal salt

The crystal structure as determined from the single crystal X-ray diffraction is given.

FIG. 9: DSC analysis of crystalline form of Memantine-HTB salt

The DSC analysis of the crystalline form of Memantine-HTB salt are shown measured as described in Example 3.

FIG. 10: TG analysis of crystalline form of Memantine-HTB salt

The TG analysis of the crystalline form of Memantine-HTB salt are shown measured as described in Example 3.

FIG. 11: Powder X-ray diffraction pattern of crystalline form of Memantine-HTB salt (XRPD).

The powder X-ray diffraction pattern of the crystalline form of Memantine-HTB salt is shown measured as described in Example 3.

FIG. 12: Crystal structure of the Memantine-HTB salt

The crystal structure as determined from the single crystal X-ray diffraction is given.

FIG. 13: DSC analysis of crystalline form of the Memantine-(S)-Ibuprofen salt

The DSC analysis of the crystalline form of Memantine-(S)-Ibuprofen salt shown is measured as described in Example 4.

FIG. 14: TG analysis of crystalline form of the Memantine-(S)-Ibuprofen salt

The TG analysis of the crystalline form of Memantine-(S)-Ibuprofen salt shown is measured as described in Example 4.

FIG. 15: Powder X-ray diffraction pattern of crystalline form of the Memantine-(S)-Ibuprofen salt (XRPD)

The powder X-ray diffraction pattern of the crystalline form of Memantine-(S)-Ibuprofen salt shown is measured as described in Example 4.

FIG. 16: Crystal structure of the Memantine-(S)-Ibuprofen salt

The crystal structure as determined from the single crystal X-ray diffraction is given.

FIG. 17: DSC analysis of crystalline form of the Memantine-Diclofenac salt

The DSC analysis of the crystalline form of Memantine-Diclofenac salt shown is measured as described in Example 5.

FIG. 18: TG analysis of crystalline form of the Memantine-Diclofenac salt

The TG analysis of the crystalline form of the Memantine-Diclofenac salt shown is measured as described in Example 5.

FIG. 19: Powder X-ray diffraction pattern of crystalline form of the Memantine-Diclofenac salt (XRPD)

The powder X-ray diffraction pattern of the crystalline form of Memantine-Diclofenac salt shown is measured as described in Example 5.

FIG. 20: Crystal structure of the Memantine-Diclofenac salt

The crystal structure as determined from the single crystal X-ray diffraction is given.

FIG. 21: DSC analysis of crystalline form of the Memantine-Acetylsalicylic acid salt

The DSC analysis of the crystalline form of Memantine-Acetylsalicylic acid salt shown is measured as described in Example 6.

FIG. 22: TG analysis of crystalline form of the Memantine-Acetylsalicylic acid salt

The TG analysis of the crystalline form of the Memantine-Acetylsalicylic acid salt shown is measured as described in Example 6.

FIG. 23: Powder X-ray diffraction pattern of crystalline form of the Memantine-Acetylsalicylic acid salt (XRPD)

The powder X-ray diffraction pattern of the crystalline form of Memantine-Acetylsalicylic acid salt shown is measured as described in Example 6.

FIG. 24: Crystal structure of the Memantine-Acetylsalicylic acid salt

The crystal structure as determined from the single crystal X-ray diffraction is given.

FIG. 25: DSC analysis of crystalline Form A of Memantine(R)-Flurbiprofen or racemic Memantine-Flurbiprofen (1:1) salt

The DSC analysis of the crystalline Form A of Memantine(R)-Flurbiprofen or racemic Memantine-Flurbiprofen (1:1) salt shown is measured as described in Example 7.

FIG. 26: TG analysis of crystalline form of crystalline of Form A of Memantine(R)-Flurbiprofen or Memantine-(rac)-Flurbiprofen (1:1) salt

The TG analysis of crystalline Form A of Memantine(R)-Flurbiprofen or Memantine-(rac)-Flurbiprofen (1:1) salt shown is measured as described in Example 7.

FIG. 27: Powder X-ray diffraction pattern of crystalline Form A of Memantine(R)-Flurbiprofen or racemic Memantine-Flurbiprofen (1:1) salt (XRPD)

The powder X-ray diffraction pattern of crystalline Form A of Memantine(R)-Flurbiprofen or racemic Memantine-Flurbiprofen (1:1) salt shown is measured as described in Example 7.

FIG. 28: Crystal structure of crystal line Form A of Memantine-(R)-Flurbiprofen (1:1) salt

The crystal structure as determined from the single crystal X-ray diffraction is given.

FIG. 29: DSC analysis of crystalline Form B of Memantine-(rac)-Flurbiprofen (1:1) salt

The DSC analysis of crystalline Form B of Memantine-(rac)-Flurbiprofen (1:1) salt shown is measured as described in Example 8.

FIG. 30: TG analysis of crystalline Form B of Memantine-(rac)-Flurbiprofen (1:1) salt

The TG analysis of crystalline Form B of Memantine-(rac)-Flurbiprofen (1:1) salt shown is measured as described in Example 8.

FIG. 31: Powder X-ray diffraction pattern of crystalline Form B of Memantine-(rac)-Flurbiprofen (1:1) salt (XRPD)

The powder X-ray diffraction pattern of crystalline Form B of Memantine-(rac)-Flurbiprofen (1:1) salt shown is measured as described in Example 8.

FIG. 32: DSC analysis of crystalline Form C of Memantine-(rac)-Flurbiprofen (1:1) salt

The DSC analysis of crystalline Form C of Memantine-(rac)-Flurbiprofen (1:1) salt shown is measured as described in Example 9.

FIG. 33: TG analysis of crystalline Form C of Memantine-(rac)-Flurbiprofen (1:1) salt

The TG analysis of crystalline Form C of Memantine-(rac)-Flurbiprofen (1:1) salt shown is measured as described in Example 9.

FIG. 34: Powder X-ray diffraction pattern of crystalline Form C of Memantine-(rac)-Flurbiprofen (1:1) salt (XRPD)

The powder X-ray diffraction pattern of crystalline Form C of Memantine-(rac)-Flurbiprofen (1:1) salt shown is measured as described in Example 9.

### EXAMPLE

### Example 1

### Preparation of Memantine-(S)-Naproxen Salt

To an assay tube containing Naproxen (100 mg, 0.43 mmol) dissolved in methanol (1.4 mL), was added at room temperature Memantine (78 mg, 0.43 mmol, 1 eq.) diluted with methanol (1 mL). A complete dissolution was obtained in an exothermic reaction. The solvent was evaporated without stirring at room temperature under atmospheric pressure. After complete evaporation, the salt of Memantine-Naproxen 1:1 was obtained as white crystals (178 mg, quantitative yield).

Good quality single crystals were obtained.

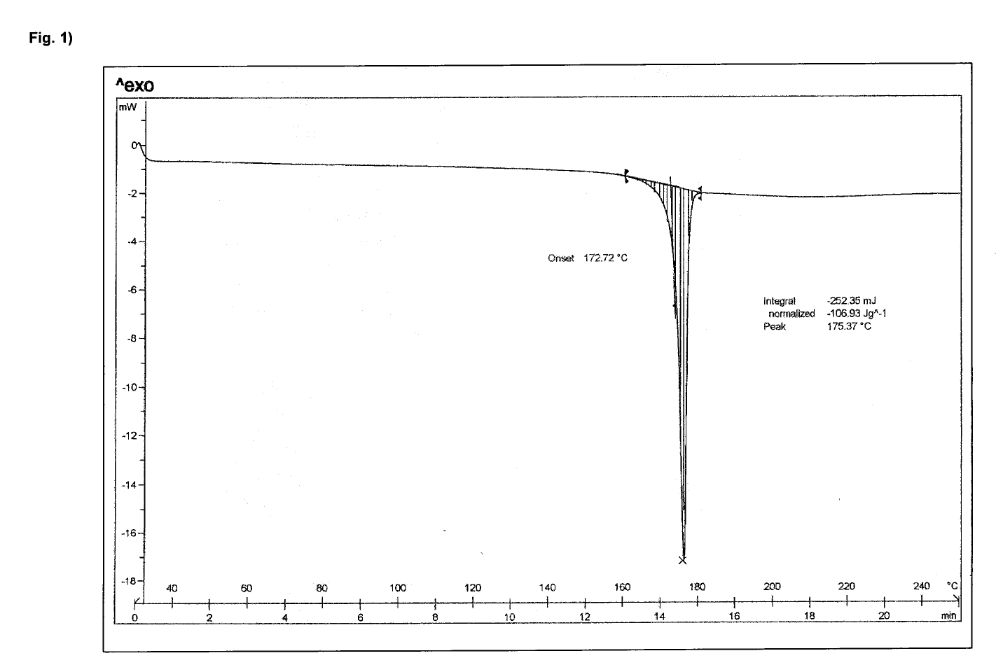

This product was fully characterized by ¹HNMR, FTIR, X-ray diffraction, and melting point (see FIGS. 1 to 3).

### FT-IR Spectrum

The FTIR spectra were recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 2946.6 (m), 2906.3 (m), 2863.6 (m), 2848.4 (m), 1632.5 (m), 1604.4 (m), 1553.2 (s), 1536.4 (s), 1378.4 (s), 1211.2 (s), 1036.3 (w), 857.6 (w), 814.35 (w).

### ¹H-NMR Spectrum

Proton nuclear magnetic resonance analyses were recorded in deuterated chloroform (CDCl₃) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum in D-chloroform at 400 MHz shows peaks at 7.63 (d, J=8.6 Hz, 1H); 7.62 (s, 1H); 7.58 (d, J=8.6 Hz, 1H); 7.46 (d, J=8.6 Hz, 1H); 7.10-7.02 (m, 2H); 3.88 (s, 3H); 3.60 (q, J=7.2 Hz, 1H); 1.90-1.83 (m, 1H); 1.47 (d, J=7.2 Hz, 3H); 1.41 (s, 2H); 1.32-1.13 (m, 4H); 1.12-0.96 (m, 4H); 0.91 (d, J=12.4 Hz, 1H); 0.79 (d, J=12.4 Hz, 1H); 0.63 (s, 6H)

### DSC Analysis (see FIG. 1)

DSC analyses were recorded with a Mettler DSC822^{e}. A sample of 2.3600 mg was weighed into a 40 μL aluminium crucible with a pinhole lid and was heated, under nitrogen (50 mL/min), at 10° C./min from 30 to 250° C.

The novel type of crystal of the present invention is characterized in that the endothermic sharp peak corresponding to the melting point has an onset at 172.72° C. (fusion enthalpy −106.93 J/g), measured by DSC analysis (10° C./min), see FIG. 1.

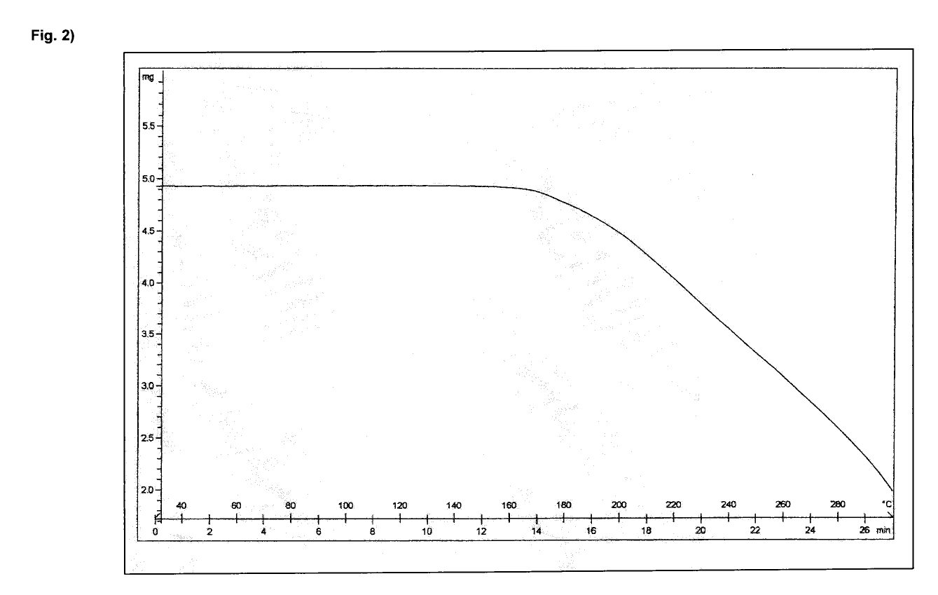

### TG Analysis (see FIG. 2)

Thermogravimetric analyses were recorded with a thermogravimetric analyzer Mettler TGA/SDTA851^{e}. A sample of 4.9178 mg was weighed into a 70 μL alumina crucible with a pinhole lid, and was heated at 10° C./min from 30 to 300° C., under nitrogen (50 mL/min).

The TG analysis of this crystalline form according to the invention shows no weight loss at temperatures lower than the melting point (see FIG. 2).

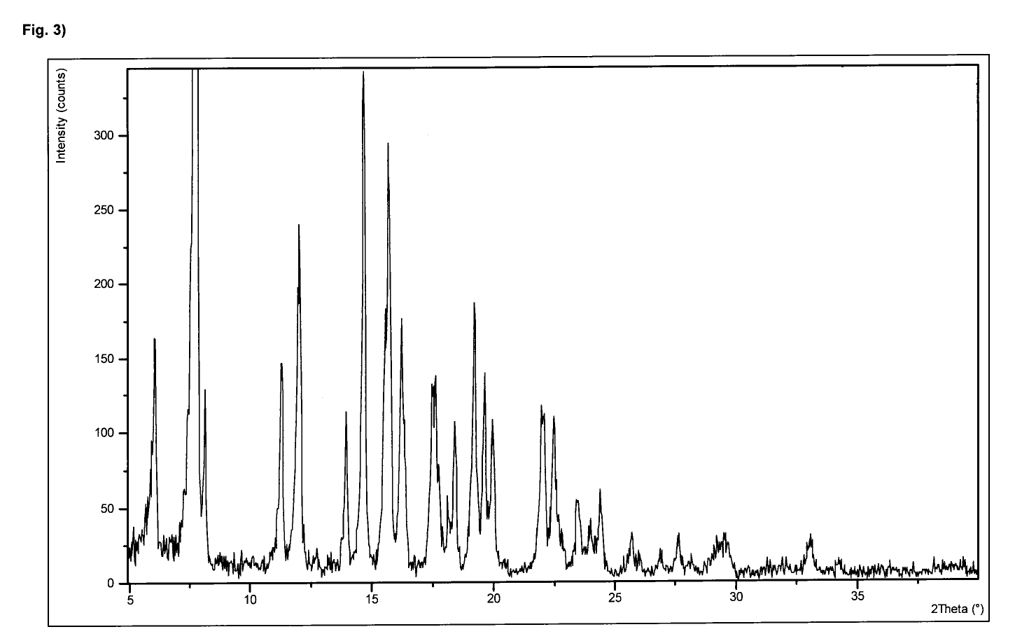

### Powder X-Ray Diffraction Pattern (XRPD) (FIG. 3)

XRPD analysis was performed using a Philips X'Pert diffractometer with Cu Kα radiation in Bragg-Brentano geometry. The system is equipped with a proportional detector. The measurement parameters were as follows: the range of 2θ was 3° to 40° at a scan rate of 1.8° per minute (see FIG. 3).

List of Selected Peaks:

| 2θ (°) | d (Å) | I (%) |
|---|---|---|
| 6.06 | 14.58 | 15 |
| 7.77 | 11.38 | 100 |
| 8.13 | 10.88 | 9 |
| 11.29 | 7.84 | 8 |
| 12.03 | 7.36 | 12 |
| 13.96 | 6.34 | 4 |
| 14.69 | 6.03 | 14 |
| 15.73 | 5.64 | 11 |
| 16.22 | 5.47 | 6 |
| 17.48 | 5.08 | 5 |
| 18.13 | 4.89 | 1 |
| 18.43 | 4.82 | 3 |
| 19.24 | 4.61 | 6 |
| 19.63 | 4.52 | 4 |
| 19.96 | 4.45 | 3 |
| 22.05 | 4.03 | 3 |
| 22.46 | 3.96 | 2 |
| 23.45 | 3.79 | 1 |
| 24.42 | 3.65 | 1 |

In addition the powder X-ray diffraction pattern of the starting products Memantine base and (S)-Naproxen were compared to the XRPD above (FIG. 3), proving formation of the salt.

Single Crystal XRD Analysis of a Single Crystal Derived from Example 1 3454.016

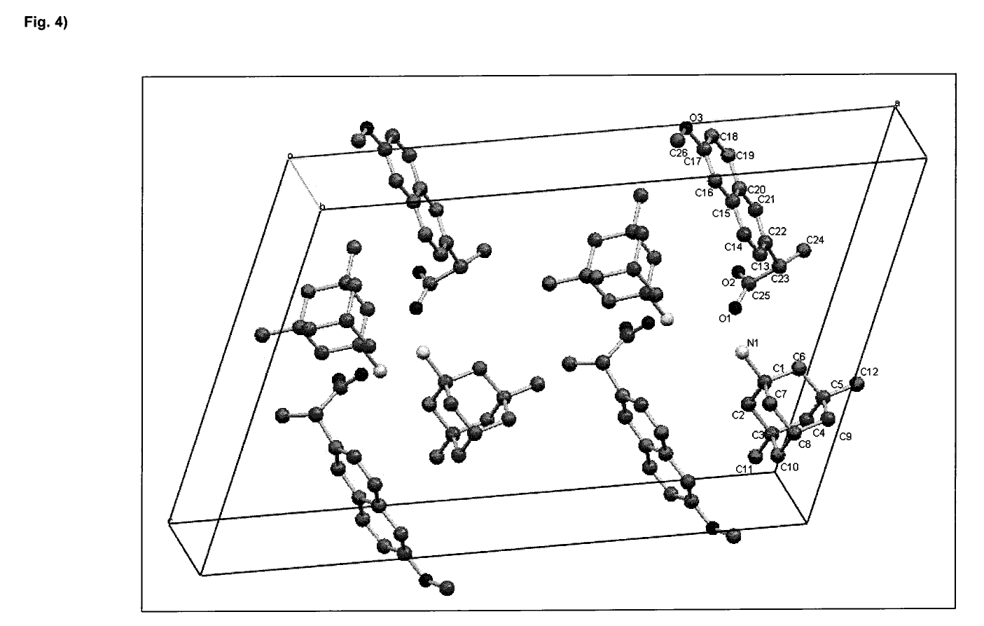

The crystal structure was determined from single crystal X-ray diffraction data (see FIG. 4). The colourless prism (0.45×0.20×0.08 mm) used was obtained from the preparation according to Example 1. Analysis was performed at room temperature using a Bruker Smart Apex diffractometer with graphite monochromated Mo_{Kα} radiation equipped with a CCD detector. Data were collected using phi and omega scans (program used: SMART 5.6). No significant decay of standard intensities was observed. Data reduction (Lorentz and polarization corrections) and absorption correction were applied (program used: SAINT 5.0). The structure was solved with direct methods and least-squares refinement of Fₒ² against all measured intensities was carried out (program used: SHELXTL-NT 6.1). All non-hydrogen atoms were refined with anisotropic displacement parameters.

Relevant Structural Data:

| Crystal system | Monoclinic |
|---|---|
| Space group: | C2 |
| a (Å) | 24.341(3) |
| b (Å) | 6.6538(7) |
| c (Å) | 15.6312(15) |
| β (°) | 109.176(2) |
| Volume (Å³) | 2391.2(5) |
| Z | 4 |
| D calc. (Mg/m³) | 1.138 |
| N. of refl. | 5080 |
| Refl. with I > 2σ(I) | 3404 |
| R (I > 2σ(I)) | 0.0701 |

The unit cell contents of this form are depicted in FIG. 4 (hydrogen atoms have been omitted for clarity; program used: Mercury 1.4.2).

Simulation of the XRPD diffractogram from single crystal data gives a diagram almost identical to the experimental one presented above.

### Example 2

### Preparation of Memantine-Triflusal Salt

To an assay tube with magnetic stirring containing Triflusal (153 mg, 0.616 mmol, 1.1 eq.) in isobutyl acetate (1 mL), was added dropwise over 5 min at room temperature 1,3-dimethyl-5-aminoadamantane (100 mg, 0.56 mmol) diluted with isobutyl acetate (1 mL). On the onset of precipitation, the mixture was stirred for 30 min at room temperature. The white solid was filtered with a sintered funnel (porosity 3) and washed with isobutyl acetate (0.8 mL). After drying at room temperature under vacuum, the salt Memantine-Triflusal 1:1 was obtained as a white solid (132 mg, 52% yield).

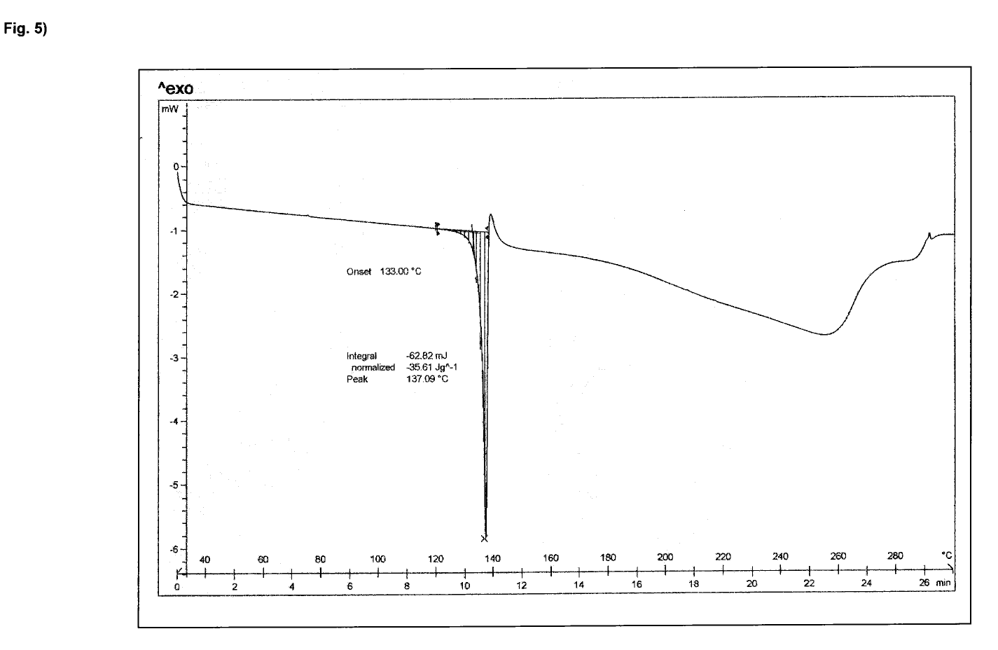

The product has been fully characterized by ¹HNMR, FTIR, X-ray diffraction, and melting point (see FIGS. 5 to 7).

### FT-IR Spectrum

The FTIR spectra were recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 2947.4 (m), 2907.5 (m), 2867.1 (m), 1777.2 (s), 1629.4 (m), 1586.5 (m), 1559.8 (s), 1385.2 (s), 1333.3 (s), 1206.5 (s), 1128.2 (s), 1108.2 (s), 943.7 (m).

### ¹H-NMR Spectrum

Proton nuclear magnetic resonance analyses were recorded in deuterated chloroform (CDCl₃) in a Varian Mercury 400 NMR spectrometer, equipped with a broadband probe ATB 1H/19F/X of 5 mm. Spectra were acquired solving 5-10 mg of sample in 0.6 mL of deuterated solvent.

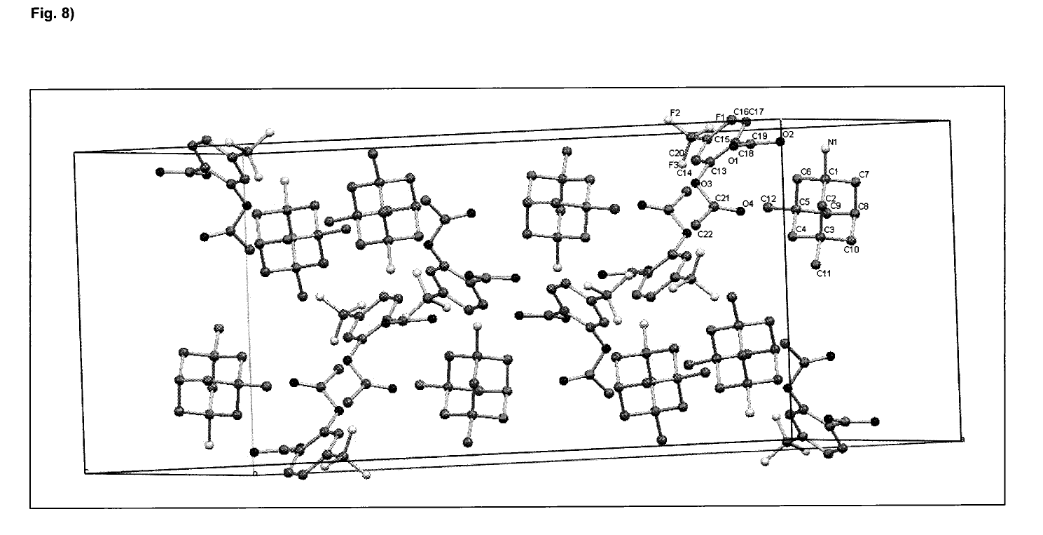

¹H NMR spectrum (see FIG. 8), in D-chloroform at 400 MHz shows peaks at 7.96 (d, J=8.1 Hz, 1H); 7.46 (dd, J=1.0 Hz, J=8.1 Hz, 1H); 7.32 (d, J=1.0 Hz, 1H); 2.27 (s, 3H); 2.14-2.06 (m, 1H); 1.66 (s, 2H); 1.45 (d, J=11.5 Hz, 2H); 1.39 (d, J=11.5 Hz, 2H); 1.29-1.17 (m, 4H); 1.09 (d, J=12.5 Hz, 1H); 0.99 (d, J=12.5 Hz, 1H); 0.76 (s, 6H).

### DSC Analysis (see FIG. 5)

DSC analyses were recorded in a Mettler Toledo DSC822e. Samples of 1-2 mg were weighted into 40 μL aluminium crucibles with a pinhole lid, and were heated under nitrogen (50 mL/min) at 10° C./min from 30 to 300° C.

The crystal shows an endothermic sharp peak corresponding to the melting point has an onset at 133.00° C. (fusion enthalpy −35.61 J/g), measured by DSC analysis (10° C./min), see FIG. 5.

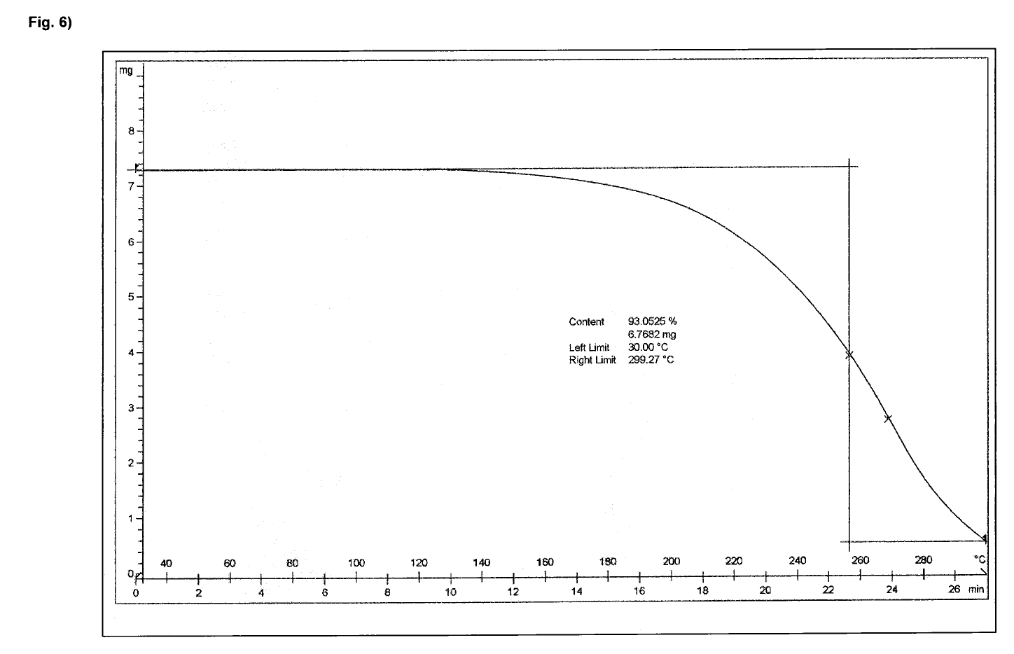

### TG Analysis (see FIG. 6).

Thermogravimetric analyses were recorded in a thermogravimetric analyzer Mettler TGA/SDTA851e. Samples of 7-8 mg were weighted into 70 μL aluminium crucibles with a pinhole lid, and heated at 10° C./min from 30 to 300° C., under nitrogen (50 mL/min).

The TG analysis of the crystalline form according to the invention shows no weight loss at temperatures lower than the melting point (see FIG. 6).

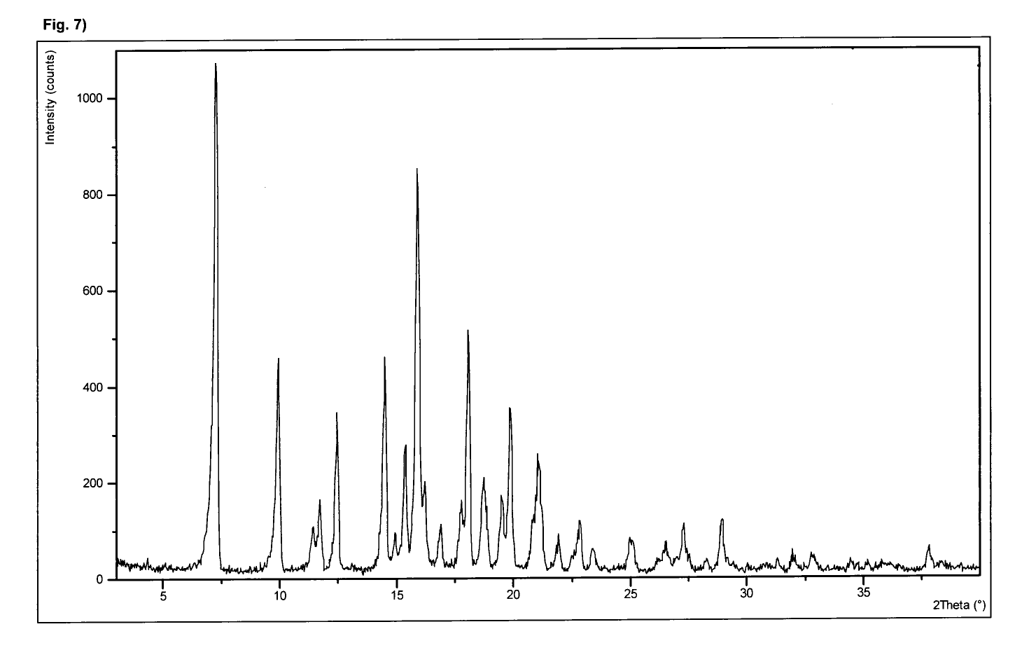

### Powder X-Ray Diffraction Pattern (XRPD) (see FIG. 7)

XRPD analysis was performed using a Philips X'Pert diffractometer with Cu K_{α} radiation in Bragg-Brentano geometry. The system is equipped with a proportional detector. The measurement parameters were as follows: the range of 2θ was 3° to 40° at a scan rate of 1.8° per minute (see FIG. 7).

List of Selected Peaks:

| 2θ (°) | d (Å) | I (%) |
|---|---|---|
| 7.30 | 12.11 | 100 |
| 9.97 | 8.88 | 29 |
| 11.41 | 7.76 | 5 |
| 11.73 | 7.55 | 8 |
| 12.48 | 7.10 | 18 |
| 14.52 | 6.10 | 21 |
| 14.95 | 5.93 | 4 |
| 15.38 | 5.76 | 12 |
| 15.94 | 5.56 | 36 |
| 16.22 | 5.46 | 8 |
| 16.90 | 5.25 | 4 |
| 17.75 | 5.00 | 5 |
| 18.09 | 4.90 | 19 |
| 18.73 | 4.74 | 7 |
| 19.52 | 4.55 | 5 |
| 19.88 | 4.47 | 12 |
| 20.81 | 4.27 | 4 |
| 21.08 | 4.22 | 8 |
| 21.97 | 4.05 | 2 |
| 22.87 | 3.89 | 3 |
| 23.41 | 3.80 | 1 |
| 25.05 | 3.55 | 2 |
| 26.48 | 3.37 | 2 |
| 27.28 | 3.27 | 3 |
| 28.91 | 3.09 | 3 |

### Single Crystal XRD Analysis of a Single Crystal of Memantine-Triflusal Salt.

Crystal structure of Memantine-Triflusal salt (1:1) has been determined from single crystal X-ray diffraction data (see FIG. 8). The colourless prism (0.34×0.10×0.04 mm) used were obtained from a liquid-liquid diffusion crystallization (chloroform-diethyl ether) with equimolar amounts of Memantine and Triflusal.

Analysis was performed at room temperature using an Oxford Diffraction Xcalibur diffractometer with graphite monochromated Cu K_{α} radiation equipped with a CCD detector. Data were collected using phi and omega scans (program used: CrysAlis CCD 1.171.32.5). No significant decay of standard intensities was observed. Data reduction (Lorentz and polarization corrections) and absorption correction were applied (program used: CrysAlis CCD 1.171.32.5). The structure was solved with direct methods and least-squares refinement of Fₒ² against all measured intensities was carried out (programs used: SIR2006 and SHELXL97). All non-hydrogen atoms were refined with anisotropic displacement parameters.

Relevant Structural Data:

| Crystal system | Monoclinic |
|---|---|
| Space group | C2/c |
| a (Å) | 30.955(4) |
| b (Å) | 13.6184(18) |
| c (Å) | 11.9378(9) |
| β (°) | 112.344(7) |
| Volume (Å³) | 4654.6(9) |
| Z | 8 |
| D calc. (Mg/m³) | 1.220 |
| N. of refl. | 3510 |
| Refl. with I > 2σ(I) | 968 |
| R (I > 2σ(I)) | 0.0613 |

The unit cell contents of this form are depicted in FIG. 8 (hydrogen atoms have been omitted for clarity; program used: Mercury 1.4.2).

Simulation of the XRPD diffractogram from single crystal data gives a diagram almost identical to the experimental one presented above.

### Example 3

### Preparation of Memantine-HTB Salt

To an assay tube containing Memantine (100 mg, 0.56 mmol) diluted with methanol (0.4 mL) was added at room temperature Triflusal (138 mg, 0.56 mmol, 1 eq.) resulting in complete dissolution (exothermic reaction). The solvent was evaporated slowly without stirring at room temperature or at 0° C. under atmospheric pressure. After complete evaporation, the salt Memantine-HTB 1:1 was obtained as colorless needles (238 mg, quantitative yield).

Good quality single crystals were obtained.

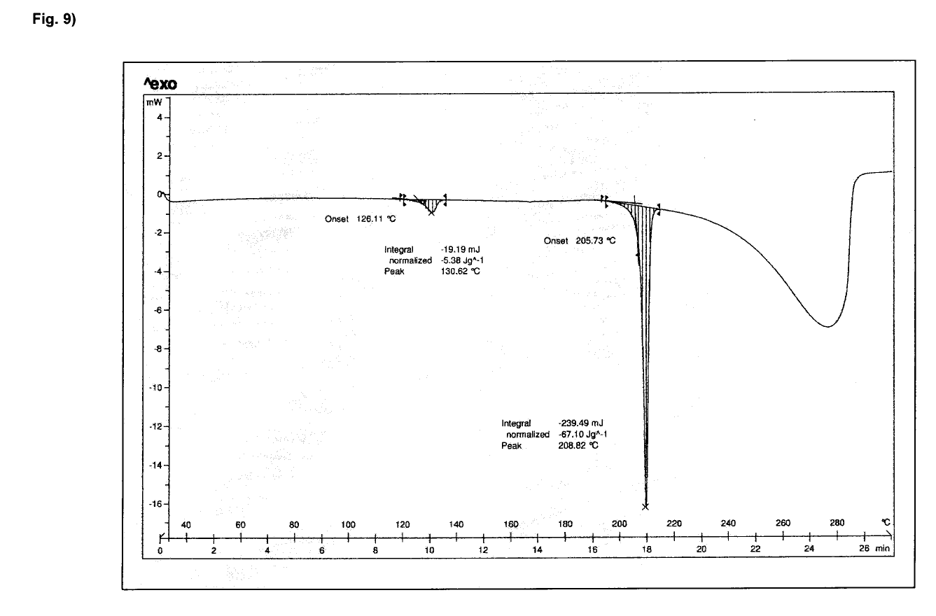

The product has been fully characterized by ¹HNMR, FTIR, X-ray diffraction, and melting point (see FIGS. 9 to 11).

### FT-IR Spectrum

FTIR spectra were recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 3178 (w, br), 2948.9 (m), 2919.2 (m), 2848.8 (m), 1592.5 (s), 1501.2 (m), 1453.7 (m), 1438.1 (s), 1389 (s), 1240 (s), 1175.2 (s), 1152 (m), 1122.4 (s), 921.3 (m) cm⁻.

### ¹H-NMR Spectrum

Proton nuclear magnetic resonance analyses were recorded in deuterated chloroform (CDCl₃) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum in D-chloroform at 400 MHz shows peaks at 7.92 (d, J=8.2 Hz, 1H); 7.22 (s, 1H); 7.07 (d, J=8.2 Hz, 1H); 2.19-2.12 (m, 1H); 1.66 (s, 2H); 1.47 (d, J=11.5 Hz, 2H); 1.40 (d, J=11.5 Hz, 2H); 1.29 (d, J=12.6 Hz, 2H); 1.19 (d, J=12.6 Hz, 2H); 1.13 (d, J=12.7 Hz, 1H); 0.95 (d, J=12.7 Hz, 1H); 0.78 (s, 6H).

### DSC Analysis (see FIG. 9)

DSC analyses were recorded with a Mettler DSC822^{e}. A sample of 3.5690 mg was weighed into 40 μL aluminium crucible with a pinhole lid and was heated, under nitrogen (50 mL/min), at 10° C./min from 30 to 300° C.

The novel type of crystal of the present invention is characterized in that the endothermic sharp peak corresponding to the melting point has an onset at 205.73° C. (fusion enthalpy −67.1 J/g), measured by DSC analysis (10° C./min) (see FIG. 9).

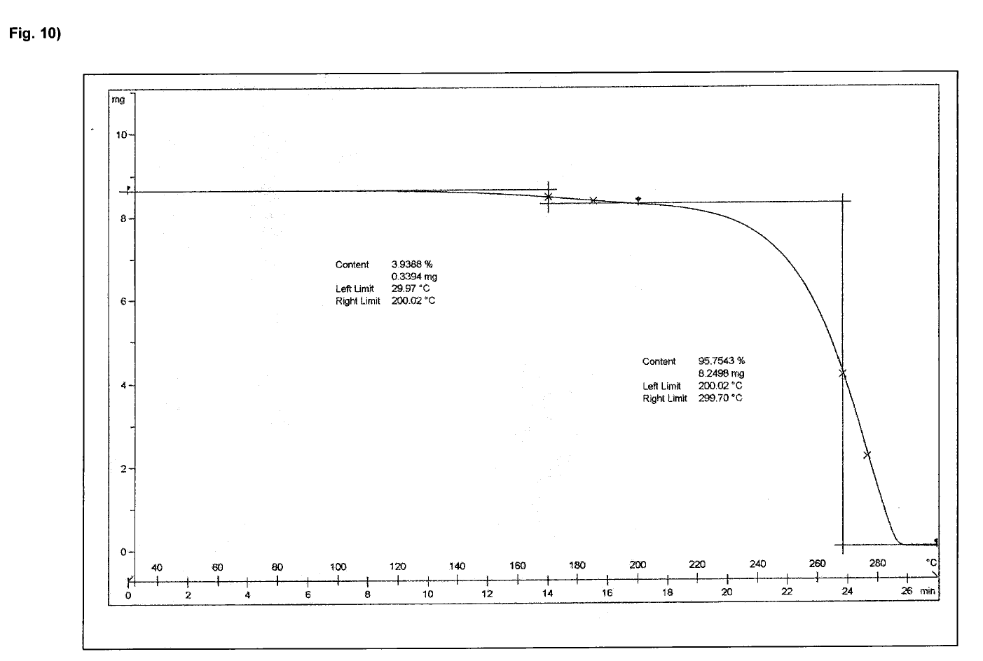

### TG Analysis (see FIG. 10)

Thermogravimetric analyses were recorded in a thermogravimetric analyzer Mettler TGA/SDTA851^{e}. A sample of 8.6156 mg was weighed into a 70 μL alumina crucible with a pinhole lid and was heated at 10° C./min from 30 to 300° C., under nitrogen (50 mL/min).

The TG analysis of the crystalline form according to the invention shows 3.94% weight loss between 30 and 200° C. corresponding to the presence of impurities derived from the preparation method (no purification) (see FIG. 10).

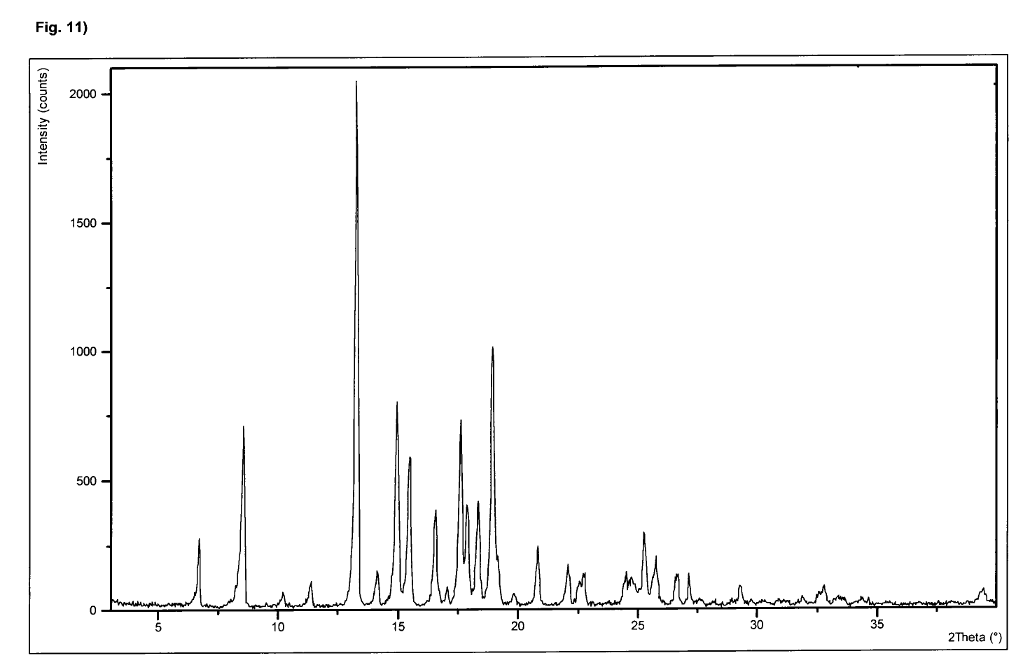

### Powder X-Ray Diffraction Pattern (see FIG. 11)

XRPD analysis was performed using a Philips X'Pert diffractometer with Cu K_{α} radiation in Bragg-Brentano geometry. The system is equipped with a proportional detector. The measurement parameters were as follows: the range of 2θ was 3° to 40° at a scan rate of 1.8° per minute (see FIG. 11).

List of Selected Peaks:

| 2θ (°) | d (Å) | I (%) |
|---|---|---|
| 6.71 | 13.18 | 26 |
| 8.57 | 10.32 | 53 |
| 10.23 | 8.65 | 4 |
| 11.39 | 7.77 | 6 |
| 13.31 | 6.65 | 100 |
| 14.15 | 6.26 | 6 |
| 14.98 | 5.91 | 34 |
| 15.51 | 5.72 | 25 |
| 16.56 | 5.35 | 14 |
| 17.07 | 5.20 | 3 |
| 17.61 | 5.04 | 25 |
| 17.90 | 4.96 | 14 |
| 18.34 | 4.84 | 15 |
| 18.95 | 4.68 | 35 |
| 19.84 | 4.48 | 2 |
| 20.82 | 4.27 | 7 |
| 22.10 | 4.02 | 5 |
| 22.54 | 3.94 | 3 |
| 22.76 | 3.91 | 3 |
| 24.50 | 3.63 | 2 |
| 25.30 | 3.52 | 7 |
| 25.76 | 3.46 | 5 |
| 26.68 | 3.34 | 3 |
| 27.15 | 3.28 | 2 |
| 29.29 | 3.05 | 2 |
| 32.80 | 2.73 | 2 |
| 39.46 | 2.28 | 1 |

### Single Crystal XRD Analysis of a Single Crystal of Memantine-HTB Salt.

The crystal structure of the Memantine-HTB salt has been determined from single crystal X-ray diffraction data. The colourless prism (0.56×0.33×0.12 mm) used was obtained from the cold evaporation of a methanol solution of equimolar amounts of Memantine and HTB.

Analysis was performed at room temperature using a Bruker Smart Apex diffractometer with graphite monochromated Mo Kα radiation equipped with a CCD detector. Data were collected using phi and omega scans (program used: SMART 5.6). No significant decay of standard intensities was observed. Data reduction (Lorentz and polarization corrections) and absorption correction were applied (program used: SAINT 5.0). The structure was solved with direct methods and least-squares refinement of Fo2 against all measured intensities was carried out (program used: SHELXTL-NT 6.1). All non-hydrogen atoms were refined with anisotropic displacement parameters.

Relevant Structural Data:

| Crystal system | Triclinic |
|---|---|
| Space group | P-1 |
| a (Å) | 7.1296(6) |
| b (Å) | 11.0891(9) |
| c (Å) | 13.5470(12) |
| α (°) | 94.453(2) |
| β (°) | 94.769(2) |
| γ (°) | 108.368(2) |
| Volume (Å³) | 1006.83(15) |
| Z | 2 |
| D calc. (Mg/m³) | 1.271 |
| N. of refl. | 4700 |
| Refl. with I > 2σ(I) | 3058 |
| R (I > 2σ(I)) | 0.0698 |

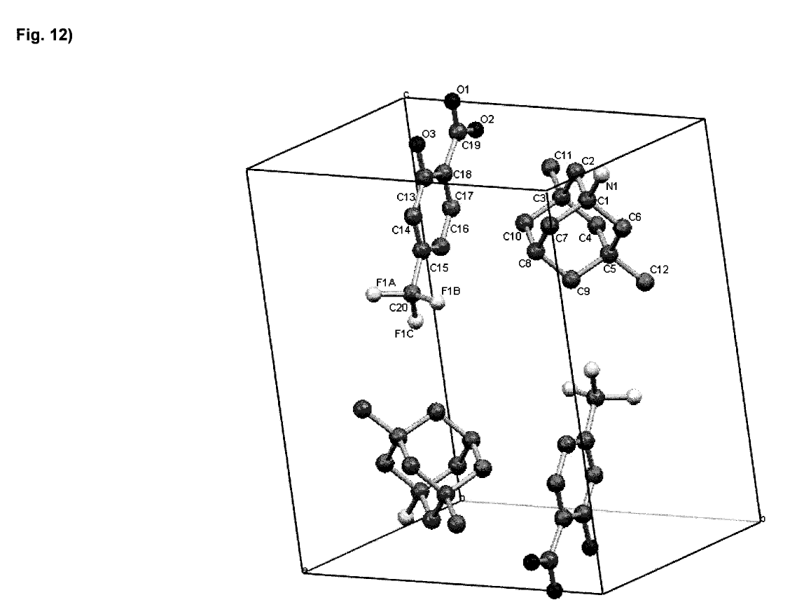

The unit cell contents of this form are depicted in FIG. 12 (hydrogen atoms have been omitted for clarity; program used: Mercury 1.4.2).

Simulation of the XRPD diffractogram from single crystal data gives a diagram almost identical diagram to the experimental one presented above.

### Example 4

### Memantine-(S)-Ibuprofen Salt

### Example 4a

### Memantine-(S)-Ibuprofen Salt

To a vial containing Memantine (80 mg, 0.44 mmol) diluted with methanol (0.5 mL) was added at room temperature (S)-Ibuprofen (92 mg, 0.44 mmol, 1 eq.) diluted with MeOH (1 mL). The solution was evaporated slowly without stirring at room temperature under atmospheric pressure. After complete evaporation, salt Memantine-(S)-Ibuprofen 1:1 was obtained as colourless needles (172 mg, quantitative yield).

### Example 4b

### Memantine-(S)-Ibuprofen Salt

To a 10 mL flask equipped with a magnetic stirrer containing (S)-Ibuprofen (218 mg, 1.06 mmol) diluted with 1.2 mL AcOEt, was added at room temperature Memantine (190 mg, 1.06 mmol, 1 eq.) diluted with AcOEt (1.6 mL). The solution was stirred at room temperature. After few seconds, a white solid precipitated. AcOEt (0.3 mL) was added to obtain a satisfactory stirring. After 10 min, the solid was filtered with a sintered funnel (porosity 3) and washed 0.1 mL AcOEt. After drying at room temperature under vacuum line, salt Memantine-(S)-Ibuprofen 1:1 was obtained as a white solid (341 mg, 84% yield).

### Example 4c

### Memantine-(S)-Ibuprofen Salt

To a 250 mL three necked flask equipped with mechanical stirrer and thermometer containing (S)-Ibuprofen (5.6 g, 27.3 mmol), was added 47 mL MIK before heating at 70° C. Then, a solution of Memantine (4.9 g, 27.3 mmol, 1 eq.) in 26 mL MIK was added over 15 min. The solution was cooled slowly. At 58° C., seeds obtained in Example 1b were added and at 55° C. crystallization started. The mixture was stirred 30 min at 55° C., 30 min at room temperature and then 1 h at 0° C.

The solid was filtered with a sinter funnel n°3 and washed with 10.5 mL MIK at 0° C. After drying at room temperature under vacuum line, salt Memantine-(S)-Ibuprofen 1:1 was obtained as a white solid (10.13 g, 96% yield).

### Characterization of the Memantine-(S)-Ibuprofen Salt

### ¹H NMR

Proton nuclear magnetic resonance analyses were recorded in deuterated chloroform (CDCl₃) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum in CDCl₃ at 400 MHz shows peaks at 7.23 (d, J=7.8 Hz, 2H); 7.03 (d, J=7.8 Hz, 1H); 3.48 (q, J=7.0 Hz, 1H); 2.40 (d, J=6.7 Hz, 2H); 2.07-2.00 (m, 1H); 1.82 (dq, J=6.7 Hz, 1H); 1.53-1.47 (m, 2H); 1.40 (d, J=7.0 Hz, 3H); 1.40-1.16 (m, 9H); 1.09-0.97 (m, 2H); 0.89 (d, J=6.7 Hz, 6H); 0.81-0.75 (m, 6H).

### IR

FTIR spectrum was recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 2954.3 (s), 2648.8 (m), 2213.2 (m), 1637.6 (s), 1547.6 (s), 1453.6 (m), 1380.3 (s), 1361.2 (s), 1282.4 (m), 1059.7 (m), 875.8 (s), 798.5 (m), 725.6 (s), 546.7 (m) cm⁻¹.

### DSC

DSC analyze was recorded with a Mettler DSC822^{e}. A sample of 4.5300 mg was weighed into 40 μL aluminium crucible with a pinhole lid and was heated, under nitrogen (50 mL/min), at 10° C./min from 30 to 200° C.

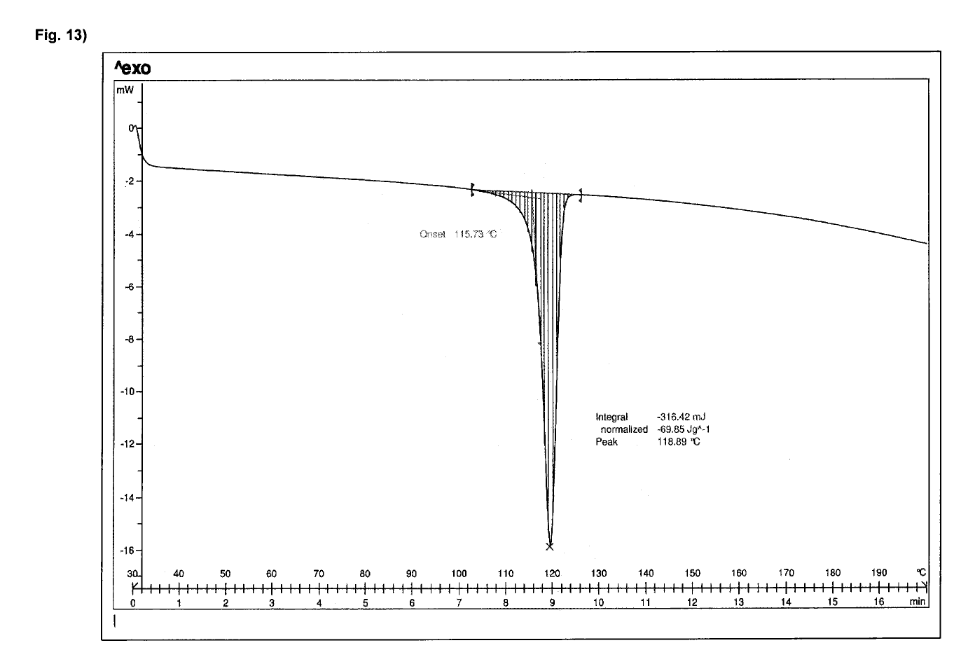

The novel type of crystal of the present invention is characterized in that the endothermic sharp peak corresponding to the melting point has an onset at 115.7° C. (fusion enthalpy −69.85 J/g), measured by DSC analysis (10° C./min) (see FIG. 13).

### TG

Thermogravimetric analysis was recorded in a thermogravimetric analyzer Mettler TGA/SDTA851^{e}. A sample of 4.0518 mg was weighed into a 70 μL alumina crucible with a pinhole lid and was heated at 10° C./min from 30 to 250° C., under nitrogen (50 mL/min).

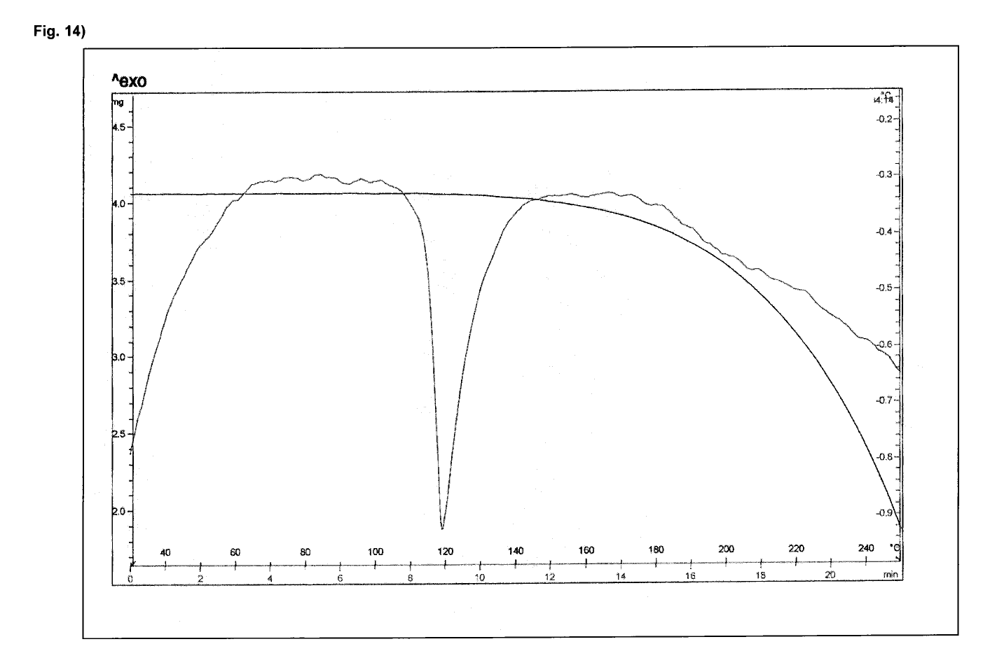

The TG analysis of this crystalline form according to the invention shows no weight loss at temperatures lower than the melting point (see FIG. 14).

### XRPD

XRPD analysis was performed using a Philips X'Pert diffractometer with Cu K_{α} radiation in Bragg-Brentano geometry. The system is equipped with a proportional detector.

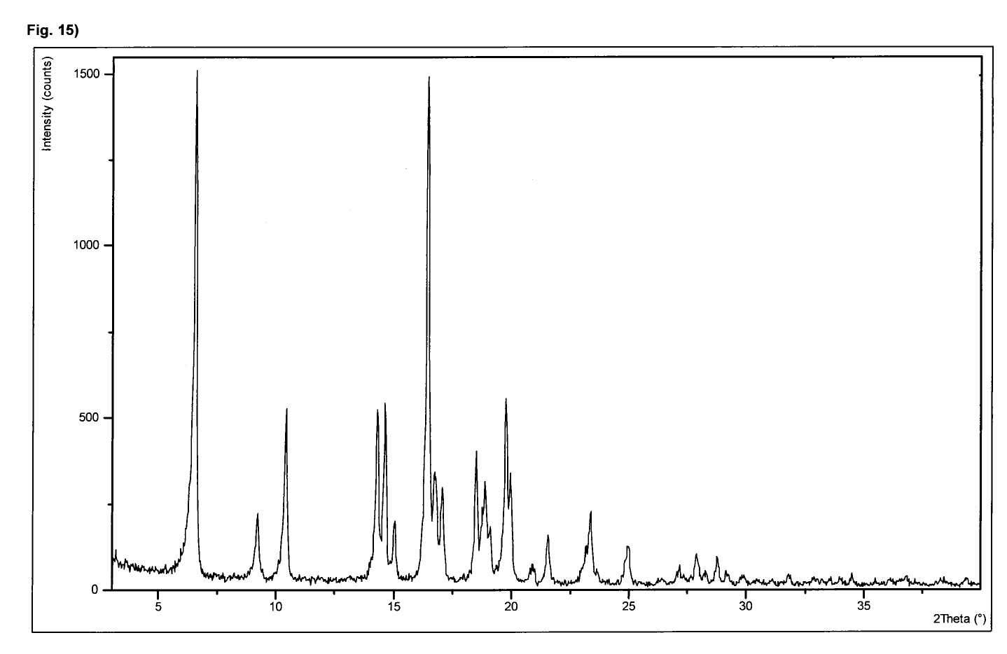

The measurement parameters were as follows: the range of 2θ was 3° to 40° at a scan rate of 1.8° per minute (see FIG. 15).

List of Selected Peaks:

| 2θ (°) | d (Å) | I (%) |
|---|---|---|
| 6.58 | 13.43 | 100 |
| 9.23 | 9.58 | 13 |
| 10.43 | 8.48 | 34 |
| 14.30 | 6.19 | 34 |
| 14.63 | 6.05 | 35 |
| 15.02 | 5.90 | 11 |
| 16.45 | 5.39 | 100 |
| 16.75 | 5.29 | 20 |
| 17.06 | 5.20 | 19 |
| 18.50 | 4.80 | 26 |
| 18.89 | 4.70 | 20 |
| 19.11 | 4.64 | 11 |
| 19.76 | 4.49 | 36 |
| 19.97 | 4.45 | 20 |
| 20.89 | 4.25 | 3 |
| 21.56 | 4.12 | 9 |
| 23.38 | 3.80 | 14 |
| 24.99 | 3.56 | 7 |
| 27.10 | 3.29 | 2 |
| 27.88 | 3.20 | 6 |
| 28.78 | 3.10 | 5 |
| 29.18 | 3.06 | 2 |
| 29.85 | 2.99 | 1 |
| 31.82 | 2.81 | 2 |
| 34.50 | 2.56 | 1 |
| 36.69 | 2.45 | 1 |

### Single Crystal X-Ray Diffraction

This crystal structure has been determined from single crystal X-ray diffraction data. The colourless crystal used (0.22×0.07×0.05 mm) was obtained from the evaporation of a solution in isopropanol of equimolar amounts of Memantine and (S)-Ibuprofen.

Analysis was performed at room temperature using an Oxford Diffraction Xcalibur Gemini diffractometer with Cu K_{α} radiation equipped with a CCD detector. The intensities were measured using the oscillation method (program used: CrysAlis CCD 1.171.32.5). No significant decay of standard intensities was observed. Data reduction (Lorentz and polarization corrections) and absorption correction were applied (program used: CrysAlis RED 1.171.32.5). The structure was solved with direct methods and full-matrix least-squares refinement of Fₒ² was carried out (programs used: SIR2006 and SHELXL97). All non-hydrogen atoms were refined with anisotropic displacement parameters.

Relevant Structural Data:

| Crystal system | Orthorhombic |
|---|---|
| Space group | P2₁2₁2₁ |
| a (Å) | 6.5700(4) |
| b (Å) | 18.9562(16) |
| c (Å) | 19.1995(19) |
| Volume (Å³) | 2391.1(3) |
| Z | 4 |
| D calc. (Mg/m³) | 1.071 |
| N. of refl. | 3898 |
| Refl. with I > 2σ(I) | 1777 |
| R (I > 2σ(I)) | 0.0415 |

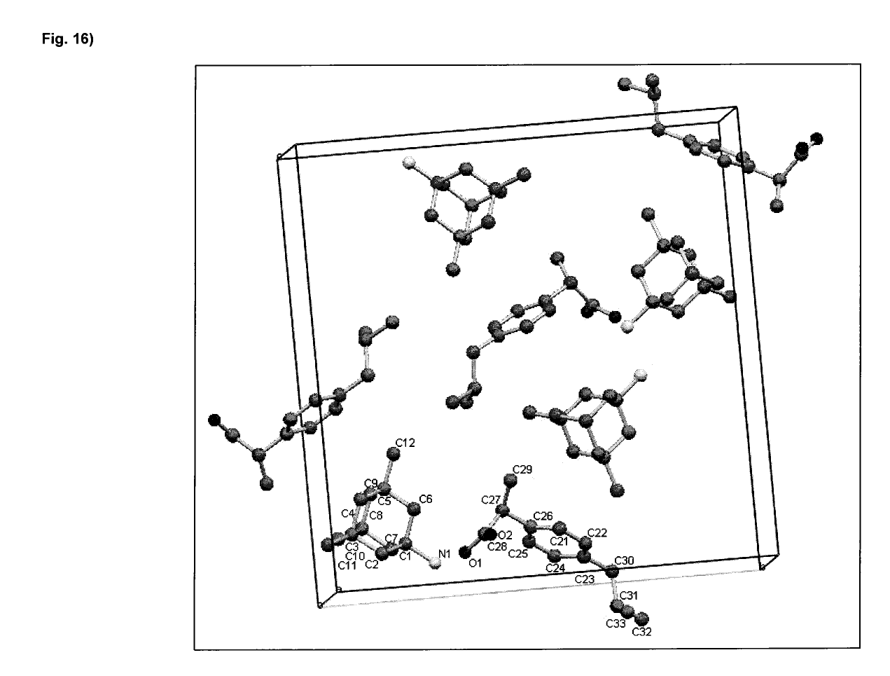

The unit cell contents of this form are depicted in FIG. 16 (hydrogen atoms have been omitted for clarity; program used: Mercury 1.4.2).

Simulation of XRPD diffractogram from single crystal data gives a diagram almost identical to the experimental one presented above.

### Example 5

### Memantine-Diclofenac Salt

### Example 5a

### Memantine-Diclofenac Salt

To an assay tube equipped with a magnetical stirrer containing Diclofenac (83 mg, 0.28 mmol) suspended in heptane (0.3 mL), was added at room temperature Memantine (50 mg, 0.28 mmol, 1 eq.) diluted with heptane (0.7 mL). Then, the mixture is stirred overnight at room temperature.

The white solid was filtered with a sintered funnel (porosity 3) and was washed with heptane (0.5 mL). After drying at 65° C. under vacuum, salt Memantine-Diclofenac 1:1 was obtained as a white powder (105 mg, 79% yield).

### Example 5b

### Memantine-Diclofenac Salt

To a vial containing Diclofenac (83 mg, 0.28 mmol) in solution with 1 mL MeOH, was added Memantine (50 mg, 0.28 mmol, 1 eq.) diluted with methanol (1 mL) at room temperature.

The solution was evaporated slowly without stirring at room temperature under atmospheric pressure. After complete evaporation, salt Memantine-Diclofenac 1:1 was obtained as colourless crystals (133 mg, quantitative yield).

### Characterization of the Memantine-Diclofenac Salt

### ¹H NMR

Proton nuclear magnetic resonance analyses were recorded in deuterated dimethylsulfoxide (d6-DMSO) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum, in d6-DMSO at 400 MHz shows peaks at 9.78 (s br, 1H); 7.44 (d, J=7.8 Hz, 2H); 7.10-7.02 (m, 2H); 6.92 (t, J=7.4 Hz, 2H); 6.74 (t, J=7.4 Hz, 1H); 6.24 (d, J=7.8 Hz, 1H); 3.39 (s, 2H); 2.11-2.03 (m, 1H); 1.61-1.52 (m, 2H); 1.39 (d, J=11.7 Hz, 2H); 1.34 (d, J=11.7 Hz, 2H); 1.27-1.15 (m, 4H); 1.08 (d, J=12.5 Hz, 1H); 1.00 (d, J=12.5 Hz, 1H); 0.78 (s, 6H).

### IR

The FTIR spectrum was recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 3211.6 (m, br), 2946.0 (s), 2848.2 (s), 2706.5 (m), 2653.7 (m), 1632.7 (m), 1547.9 (s), 1504.1(s), 1494.6 (s), 1466.5 (s), 1452.1 (s), 1386.4 (s), 872.9 (m), 766.9 (s), 744.8 (s), 718.4 (m).

### DSC

DSC analysis was recorded with a Mettler DSC822^{e}. A sample of 1.5700 mg was weighed into a 40 μL aluminium crucible with a pinhole lid, and was heated, under nitrogen (50 mL/min), at 10° C./min from 30 to 300° C.

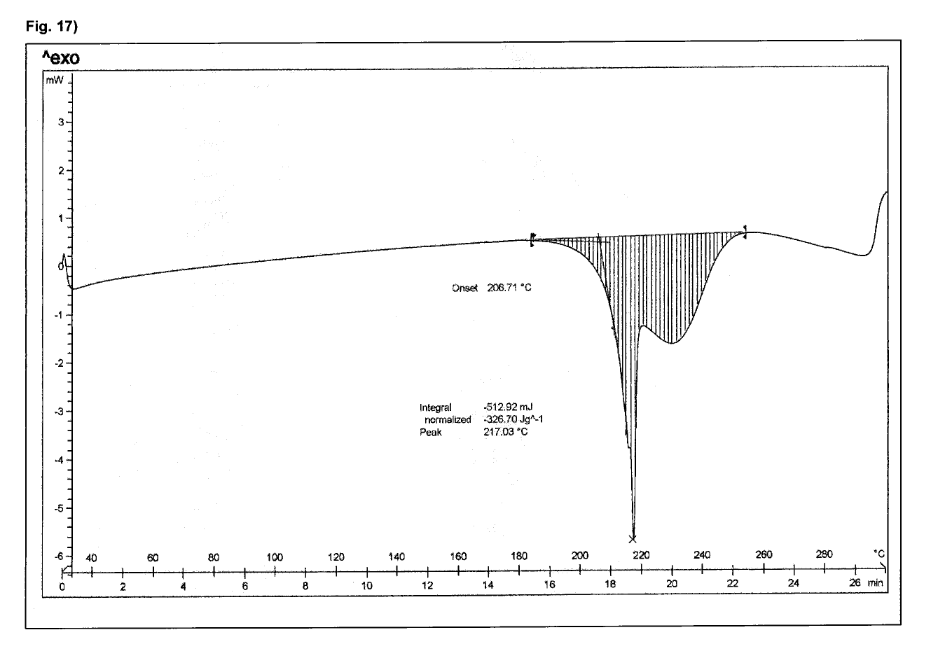

The novel type of crystal of the present invention is characterized in that the endothermic sharp peak corresponding to the melting point has an onset at 206.7° C. (fusion enthalpy+degradation enthalpy −326.7 J/g), measured by DSC analysis (10° C./min) (see FIG. 17).

### TG

Thermogravimetric analysis was recorded in a thermogravimetric analyzer Mettler TGA/SDTA851^{e}. A sample of 5.5237 mg was weighed into a 70 μL alumina crucible with a pinhole lid, and was heated at 10° C./min from 30 to 300° C., under nitrogen (50 mL/min).

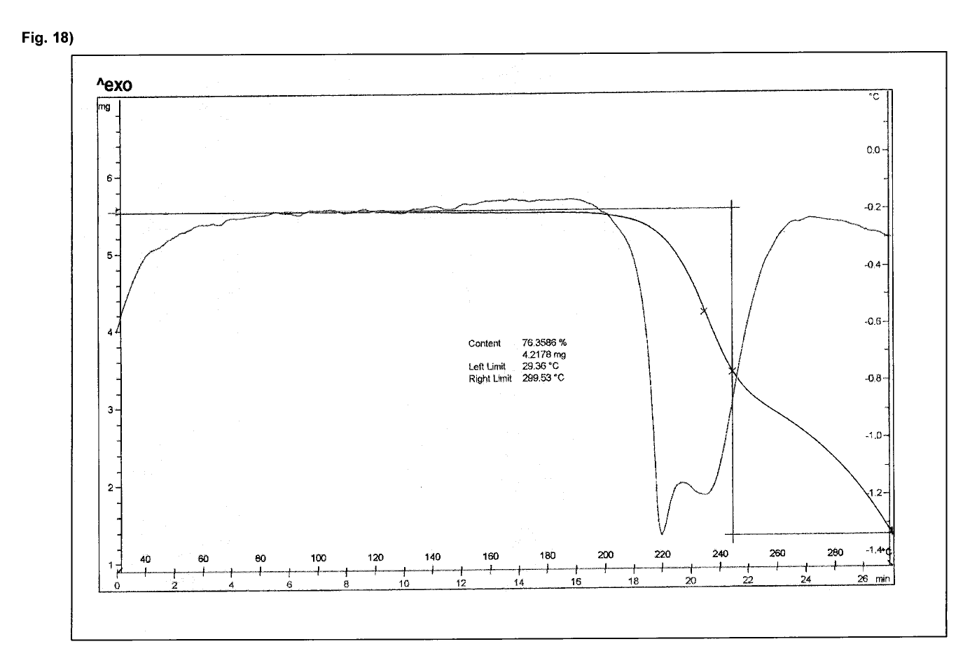

The TG analysis of this crystalline form according to the invention shows no weight loss at temperatures lower than the melting point. The loss of weight during the fusion became from the degradation (see FIG. 18).

### XRPD

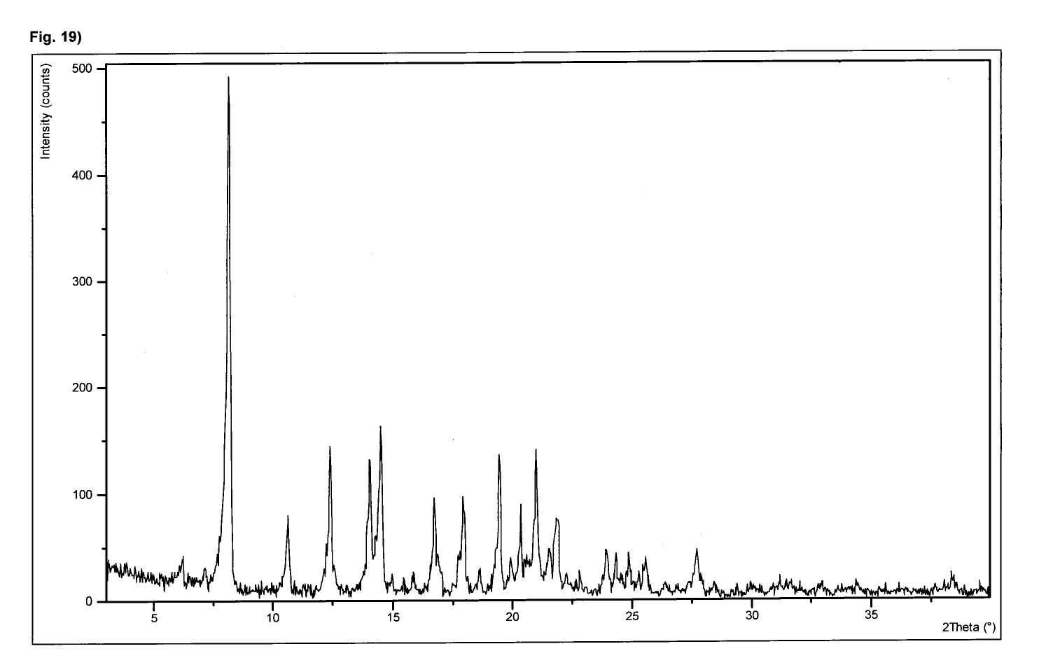

XRPD analysis was performed using a Philips X'Pert diffractometer with Cu K_{α} radiation in Bragg-Brentano geometry. The system is equipped with a proportional detector. The measurement parameters were as follows: the range of 2θ was 3° to 40° at a scan rate of 1.8° per minute (see FIG. 19).

List of Selected Peaks:

| 2θ (°) | d (Å) | I (%) |
|---|---|---|
| 8.17 | 10.82 | 100 |
| 10.61 | 8.33 | 15 |
| 12.39 | 7.14 | 28 |
| 14.04 | 6.31 | 28 |
| 14.52 | 6.10 | 33 |
| 16.72 | 5.30 | 19 |
| 17.94 | 4.94 | 18 |
| 18.61 | 4.77 | 4 |
| 19.44 | 4.57 | 28 |
| 20.99 | 4.23 | 29 |
| 21.87 | 4.06 | 15 |
| 23.92 | 3.72 | 9 |
| 24.66 | 3.61 | 3 |
| 25.56 | 3.48 | 7 |
| 27.69 | 3.22 | 8 |
| 31.38 | 2.85 | 1 |
| 38.42 | 2.34 | 3 |

### Single Crystal X-Ray Diffraction

This crystal structure has been determined from single crystal X-ray diffraction data. The colourless prismatic crystal used (0.38×0.31×0.07 mm) was obtained from the evaporation of a solution in methanol of equimolar amounts of Memantine and Diclofenac.

Analysis was performed at room temperature using an Oxford Diffraction Xcalibur Gemini diffractometer with Cu K_{α} radiation equipped with a CCD detector. The intensities were measured using the oscillation method (program used: CrysAlis CCD 1.171.32.5). No significant decay of standard intensities was observed. Data reduction (Lorentz and polarization corrections) and absorption correction were applied (program used: CrysAlis RED 1.171.32.5). The structure was solved with direct methods and full-matrix least-squares refinement of Fₒ² was carried out (programs used: SIR2006 and SHELXL97). All non-hydrogen atoms were refined with anisotropic displacement parameters.

Relevant Structural Data:

| Crystal system | Monoclinic |
|---|---|
| Space group | P2₁/n |
| a (Å) | 16.9403(1) |
| b (Å) | 6.7805(1) |
| c (Å) | 22.0373(2) |
| β (°) | 98.513(1) |
| Volume (Å³) | 2503.4(2) |
| Z | 4 |
| D calc. (Mg/m³) | 1.261 |
| N. of refl. | 4264 |
| Refl. with I > 2σ(I) | 3240 |
| R (I > 2σ(I)) | 0.0344 |

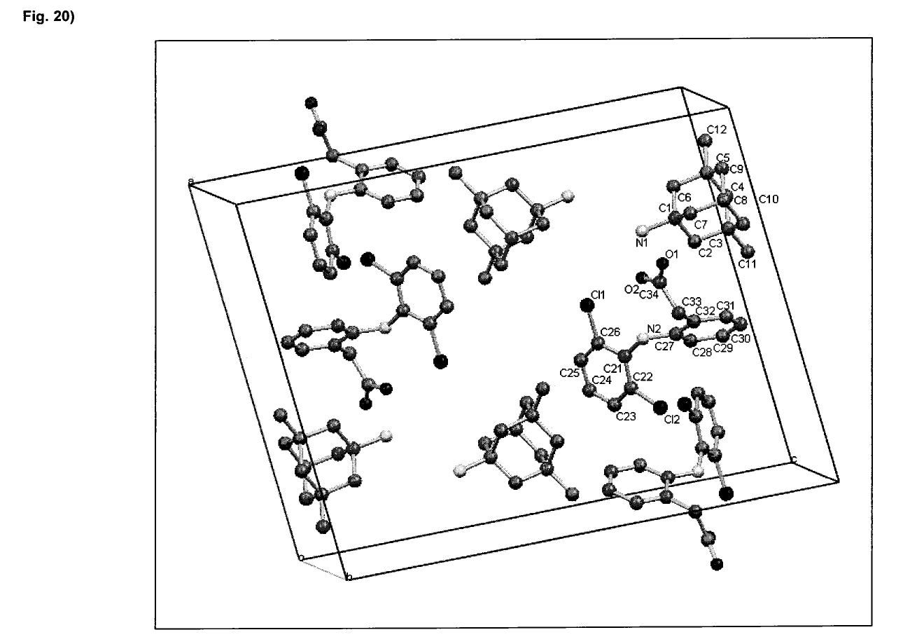

The unit cell contents of this form are depicted in FIG. 20 (hydrogen atoms have been omitted for clarity; program used: Mercury 1.4.2).

Positions of peaks in XRPD diffractogram simulated from single crystal data are almost identical to those in the experimental one presented above.

### Example 6

### Memantine-Acetylsalicylic Acid Salt

### Example 6a

### Memantine-Acetylsalicylic Acid Salt

To an assay tube equipped with a magnetical stirrer containing Acetylsalicylic acid (100 mg, 0.55 mmol) diluted with AcOiBu (1 mL), was added at room temperature Memantine (100 mg, 0.55 mmol, 1 eq.) diluted with AcOiBu (1 mL). A precipitated was obtained after few minutes and the mixture was stirred at room temperature overnight.

The white solid was filtered with a sintered funnel (porosity 3) and was washed with AcOiBu (0.4 mL). After drying at room temperature under vacuum, salt Memantine-Acetylsalicylic acid 1:1 was obtained as a white powder (172 mg, 86% yield).

### Example 6b

### Memantine-Acetylsalicylic Acid Salt

To an assay tube equipped with a magnetical stirrer containing Acetylsalicylic acid (72 mg, 0.4 mmol) diluted with ACN (0.3 mL), was added dropwise at room temperature 1,3-dimethyl-5-aminoadamantane (72 mg, 0.4 mmol, 1 eq.) diluted with ACN (0.4 mL). A precipitated was obtained after few seconds and ACN (1.5 mL) was added to obtain a satisfactory stirring.

The white solid was filtered with a sintered funnel (porosity 3) and was washed with ACN (0.2 mL). After drying at room temperature under vacuum, salt Memantine-Acetylsalicylic acid 1:1 was obtained as a white powder (111 mg, 77% yield).

### Example 6c

### Memantine-Acetylsalicylic Acid Salt

To a 10 mL flask equipped with magnetical stirrer containing Acetylsalicylic acid (200 mg, 1.1 mmol), was added 1 mL AcOiBu before cooling at 0° C. Then, a solution of Memantine (200 mg, 1.1 mmol, 1 eq.) in 2 mL AcOiBu was added over 3 h and the suspension was stirred 30 min at 0° C.

The solid was filtered with a sinter funnel n° 3 and washed with 0.3 mL AcOiBu at 0° C. After drying at room temperature under vacuum line, salt Memantine-Acetylsalicylic acid 1:1 was obtained as a white solid (286 mg, 72% yield).

### Characterization of the Memantine-Acetylsalicylic Acid Salt

### ¹H NMR

Proton nuclear magnetic resonance analyses were recorded in deuterated chloroform (CDCl₃) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum in d-chloroform at 400 MHz shows peaks at 7.92 (dd, J=1.6 Hz, J=7.4 Hz, 1H); 7.40 (dt, J=1.6 Hz, J=8.2 Hz, 1H); 7.21 (dt, J=1.2 Hz, J=7.4 Hz, 1H); 7.02 (dd, J=1.2 Hz, J=8.2 Hz, 1H); 2.25 (s, 3H); 2.11-2.04 (m, 1H); 1.72-1.64 (m, 2H); 1.49 (d, J=11.7 Hz, 2H); 1.42 (d, J=11.7 Hz, 2H); 1.27-1.14 (m, 4H); 1.04 (d, J=12.5 Hz, 1H); 0.98 (d, J=12.5 Hz, 1H); 0.75 (s, 6H).

### IR

The FTIR spectrum was recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 2909.6 (s), 2638.3 (m), 1765.8 (s), 1751.8 (s), 1622.9 (s), 1606.3 (s), 1590.0 (s), 1551.5 (s), 1386.0 (s), 1368.5 (s), 1218.8 (s), 1196.2 (s), 1091.2 (m), 918.3 (m), 750.1 (m).

### DSC

DSC analysis was recorded with a Mettler DSC822^{e}. A sample of 4.0060 mg was weighed into a 40 μL aluminium crucible with a pinhole lid and was heated, under nitrogen (50 mL/min), at 10° C./min from 30 to 200° C.

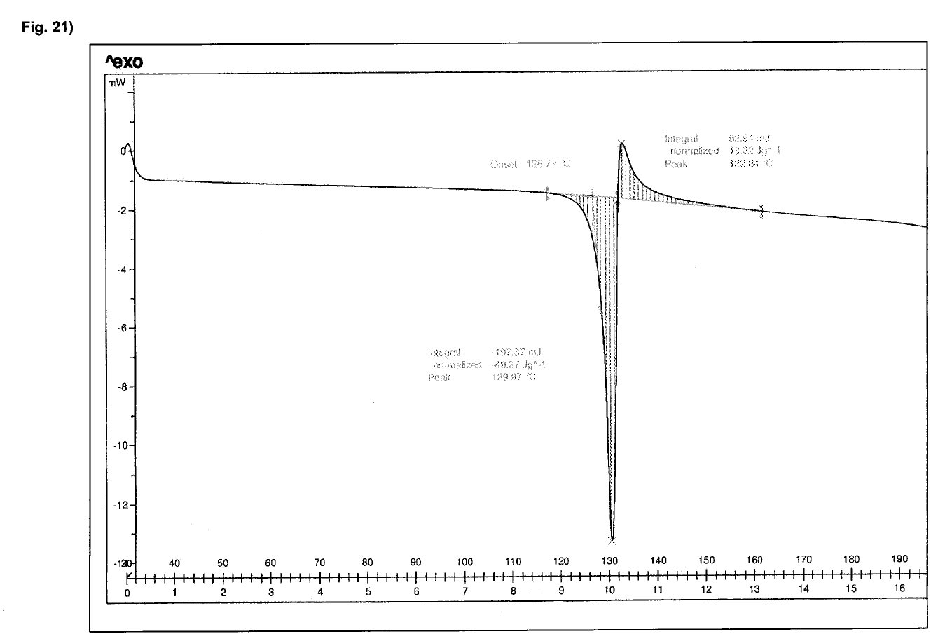

The novel type of crystal of the present invention is characterized in that the endothermic sharp peak corresponding to the melting point has an onset at 126.8° C. (fusion enthalpy −49.3 J/g), measured by DSC analysis (10° C./min), see FIG. 21.

### TG

Thermogravimetric analysis was recorded with a thermogravimetric analyzer Mettler TGA/SDTA851^{e}. A sample of 5.4594 mg was weighed into a 70 μL alumina crucible with a pinhole lid, and was heated at 10° C./min from 30 to 200° C., under nitrogen (50 mL/min).

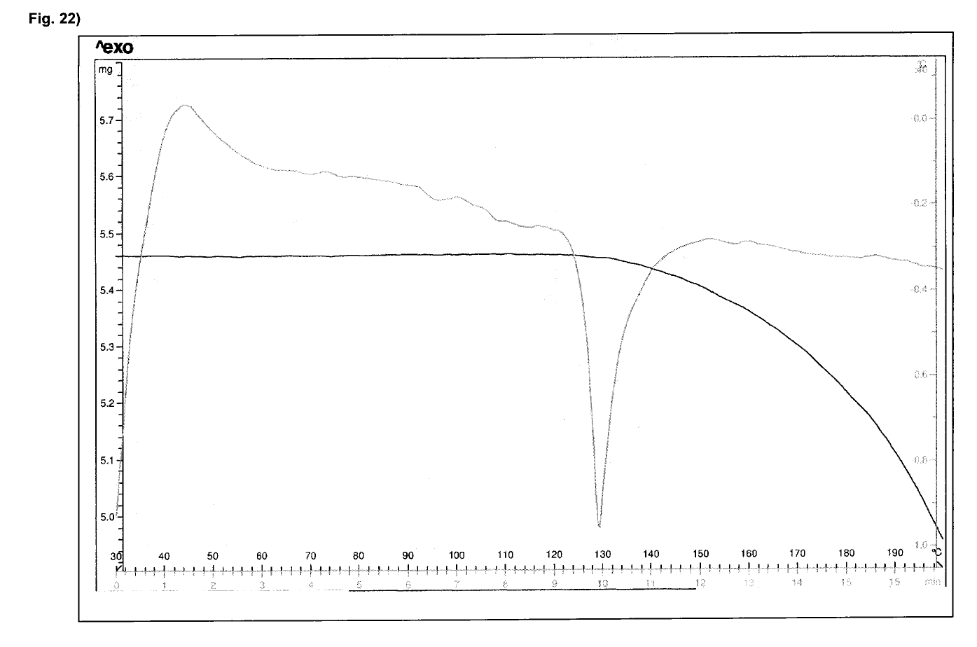

The TG analysis of this crystalline form according to the invention shows no weight loss at temperatures lower than the melting point (see FIG. 22).

### XRPD

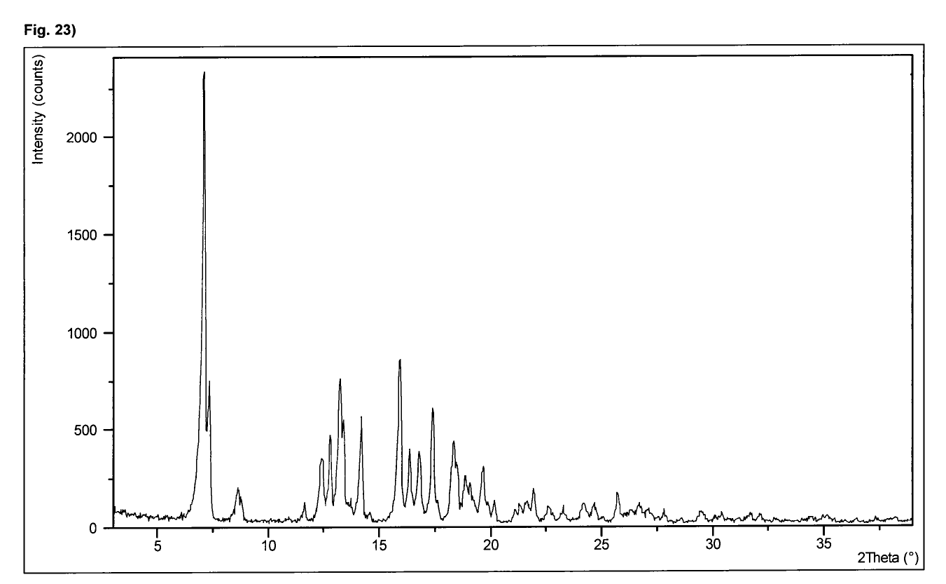

XRPD analysis was performed using a Philips X'Pert diffractometer with Cu K_{α} radiation in Bragg-Brentano geometry. The system is equipped with a proportional detector. The measurement parameters were as follows: the range of 2θ was 3° to 39° at a scan rate of 1.8° per minute (see FIG. 23).

List of Selected Peaks:

| 2θ (°) | d (Å) | I (%) |
|---|---|---|
| 7.11 | 12.44 | 100 |
| 7.35 | 12.02 | 30 |
| 8.62 | 10.25 | 7 |
| 11.63 | 7.60 | 3 |
| 12.43 | 7.12 | 14 |
| 12.79 | 6.92 | 19 |
| 13.22 | 6.69 | 31 |
| 14.20 | 6.24 | 23 |
| 15.94 | 5.56 | 36 |
| 16.36 | 5.42 | 16 |
| 16.79 | 5.28 | 15 |
| 17.39 | 5.10 | 25 |
| 18.32 | 4.84 | 17 |
| 18.51 | 4.79 | 11 |
| 18.84 | 4.71 | 10 |
| 19.67 | 4.51 | 12 |
| 20.16 | 4.40 | 5 |
| 21.95 | 4.05 | 6 |
| 22.59 | 3.93 | 3 |
| 23.25 | 3.83 | 2 |
| 24.17 | 3.68 | 4 |
| 24.67 | 3.61 | 4 |
| 25.72 | 3.46 | 6 |
| 26.70 | 3.34 | 4 |
| 27.83 | 3.21 | 2 |

### Single Crystal X-Ray Diffraction

This crystal structure has been determined from single crystal X-ray diffraction data. The colourless crystal used (0.38×0.10×0.05 mm) was obtained from the evaporation of a solution in DMSO of equimolar amounts of Memantine and Acetylsalicylic acid.

Analysis was performed at 100 K using an Oxford Diffraction Xcalibur Nova diffractometer with Cu K_{α} radiation equipped with a CCD detector. The intensities were measured using the oscillation method (program used: CrysAlis CCD 1.171.32.37). No significant decay of standard intensities was observed. Data reduction (Lorentz and polarization corrections) and absorption correction were applied (program used: CrysAlis RED 1.171.32.37). The structure was solved with direct methods and full-matrix least-squares refinement of Fₒ² was carried out (programs used: SIR2006 and SHELXL97). All non-hydrogen atoms were refined with anisotropic displacement parameters.

Relevant Structural Data:

| Crystal system | Triclinic |
|---|---|
| Space group | P-1 |
| a (Å) | 11.5725(8) |
| b (Å) | 14.4971(12) |
| c (Å) | 14.5226(10) |
| α (°) | 115.676(7) |
| β (°) | 105.591(6) |
| γ (°) | 101.747(6) |
| Volume (Å³) | 1966.1(2) |
| Z | 4 |
| D calc. (Mg/m³) | 1.214 |
| N. of refl. | 6072 |
| Refl. with I > 2σ(I) | 4672 |
| R (I > 2σ(I)) | 0.0827 |

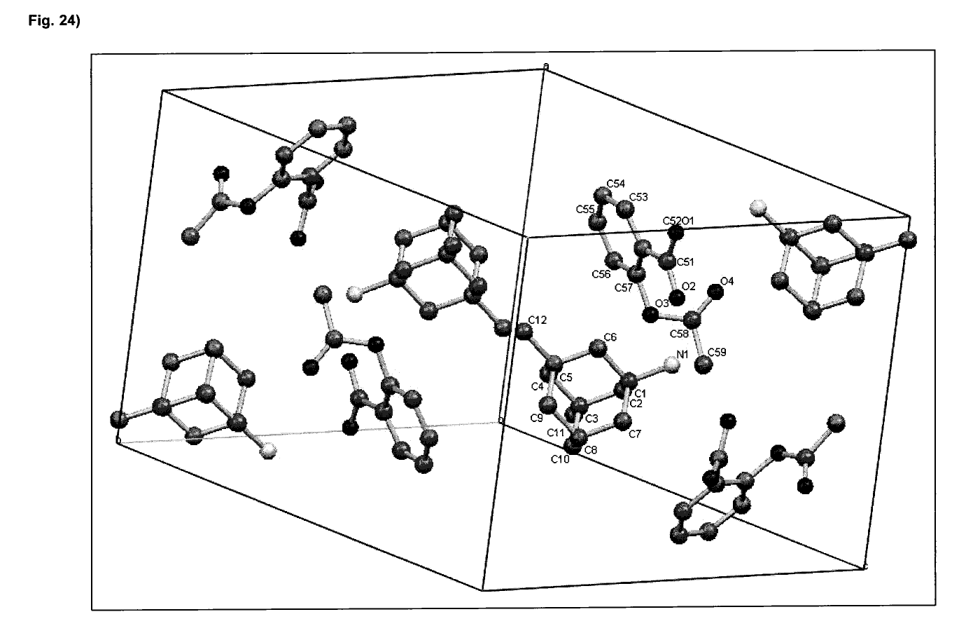

The unit cell contents of this form are depicted in FIG. 24 (hydrogen atoms have been omitted for clarity; program used: Mercury 1.4.2).

Simulation of XRPD diffractogram from single crystal data (measured at 100 K) gives a diagram almost identical to the experimental one presented above (measured at room temperature).

### Example 7

### Form A: Memantine-(R)-Flurbiprofen or Memantine-(rac)-Flurbiprofen (1:1)

### Example 7a

### Memantine-(R)-Flurbiprofen Salt Form A

To a vial containing Memantine (44 mg, 0.25 mmol), was added at room temperature (R)-Flurbiprofen (60 mg, 0.25 mmol, 1 eq.) diluted with ACN (0.4 mL).

The solution was evaporated slowly without stirring at room temperature under atmospheric pressure. After complete evaporation, salt form A Memantine-(R)-Flurbiprofen 1:1 was obtained as a white solid (104 mg, quantitative yield).

### Example 7b

### Memantine-(R)-Flurbiprofen Salt Form A

To a two necked 10 mL flask equipped with magnetical stirrer and thermometer containing (R)-Flurbiprofen (250 mg, 1.02 mmol), was added 1.4 mL IPA before heating until obtain complete dissolution (80° C.). Then, a solution of Memantine (183 mg, 1.02 mmol, 1 eq.) in 1.2 mL IPA was added slowly. The solution was cooled slowly. At 65° C., seeds obtained following Example 4a were added and the crystallization started. The mixture was stirred 30 min at 65° C., 30 min at room temperature and then 1 h at 0° C.

The solid was filtered with a sinter funnel n° 3 and washed with 0.4 mL IPA at 0° C. After drying at room temperature under vacuum line, salt form A Memantine-(R)-Flurbiprofen 1:1 was obtained as a white solid (348 mg, 81% yield).

### Example 7c

### Memantine-(R)-Flurbiprofen Salt Form A

To a three necked 250 mL flask equipped with mechanical stirrer and thermometer containing (R)-Flurbiprofen (6.50 g, 26.61 mmol), was added AcOiBu (36.4 mL) before heating until obtain complete dissolution (90° C.). Then, a solution of 1,3-dimethyl-5-aminoadamantane (4.77 g, 26.61 mmol, 1 eq.) diluted in AcOiBu (31.2 mL) was added slowly (addition time: 10 min). The solution was cooled slowly. At 80° C., seeds obtained following Example 4a were added and the crystallization started at 76° C. The mixture was stirred 30 min at 76° C., 30 min at room temperature and then 1 h at 0° C.

The solid was filtered with a sinter funnel n° 3 and washed with 11.2 mL AcOiBu at 0° C. After drying at room temperature under vacuum line, salt form A Memantine-(R)-Flurbiprofen 1:1 was obtained as a white solid (10.84 g, 96% yield).

### Optical Rotation Form A

Optical rotation was obtained at 25° C. on a Perkin-Elmer 241 polarimeter equipped with a Na lamp operating at 589 nm. The volume of the cell was 1 mL and the length of the optical path 10 cm.

The optical rotation of the crystalline form according to the invention shows α_{D}=+10.8 (c=1, MeOH)

### Example 7d

### Memantine-(rac)-Flurbiprofen Salt Form A

To a vial containing (rac)-Flurbiprofen (50 mg, 0.20 mmol) in solution with 1 mL MIK, was added Memantine (36 mg, 0.20 mmol, 1 eq.) diluted with MIK (1 mL) at room temperature.

The solution was evaporated slowly without stirring at room temperature under atmospheric pressure. After complete evaporation, Memantine-Flurbiprofen 1:1 racemic salt was obtained as colourless crystals (86 mg, quantitative yield).

### Example 7e

### Memantine-(rac)-Flurbiprofen Salt Form A

To an assay tube equipped with a magnetical stirrer containing (rac)-Flurbiprofen (50 mg, 0.20 mmol) diluted with 0.7 mL ACN, was added dropwise at room temperature Memantine (36 mg, 0.20 mmol, 1 eq.) diluted with AcOiBu (0.8 mL). A precipitated was obtained after few minutes and the mixture was stirred at room temperature for 2 h.

The white solid was filtered with a sintered funnel (porosity 3) and was washed with ACN (0.5 mL). After drying at room temperature under vacuum, Memantine-Flurbiprofen 1:1 racemic salt was obtained as a white powder (47 mg, 55% yield).

### Example 7f

### Memantine-(rac)-Flurbiprofen Salt Form A

To an assay tube equipped with magnetical stirring containing (rac)-Flurbiprofen (50 mg, 0.20 mmol), was added at room temperature Memantine (36 mg, 0.20 mmol, 1 eq.) diluted with cyclohexane (0.7 mL). After the addition, a complete dissolution was obtained. The solution was seeded with form A and a precipitate was observed. Then, the mixture was stirred 3 h at room temperature.

The white solid was filtered with a sintered funnel (porosity 3) and was washed with cyclohexane (0.5 mL). After drying at room temperature under vacuum, Memantine-Flurbiprofen 1:1 racemic salt was obtained as a white powder (28 mg, 32% yield).

### Characterization of the Form A

### ¹H NMR

Proton nuclear magnetic resonance analyses were recorded in deuterated chloroform (CDCl₃) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum in CDCl₃ at 400 MHz shows peaks at 7.52-7.46 (m, 2H); 7.44-7.38 (m, 2H); 7.37-7.29 (m, 2H); 7.23-7.16 (m, 2H); 3.59 (q, J=7.0 Hz, 1H); 2.13-2.02 (m, 1H); 1.58-1.50 (m, 2H); 1.46 (d, J=7.0 Hz, 3H); 1.38 (d, J=11.7 Hz, 2H); 1.32 (d, J=11.7 Hz, 2H); 1.27-1.18 (m, 4H); 1.08 (d, J=12.5 Hz, 1H); 1.01 (d, J=12.5 Hz, 1H); 0.79 (s, 6H)

### IR

The FTIR spectrum was recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 2947.8 (m), 2902.6 (m), 2841.9 (m), 2646.6 (m), 1635.8 (s), 1551.8 (s), 1483.2 (m), 1455.2 (s), 1416.8 (s), 1378.9 (s), 1358.9 (s), 1315.6 (m), 1262.9 (m), 1130.6 (m), 766.0 (s), 725.7 (m), 698.0 (s).

### DSC

DSC analysis was recorded with a Mettler DSC822^{e}. A sample of 1.3690 mg was weighed into a 40 μL aluminium crucible with a pinhole lid and was heated, under nitrogen (50 mL/min), at 10° C./min from 30 to 220° C.

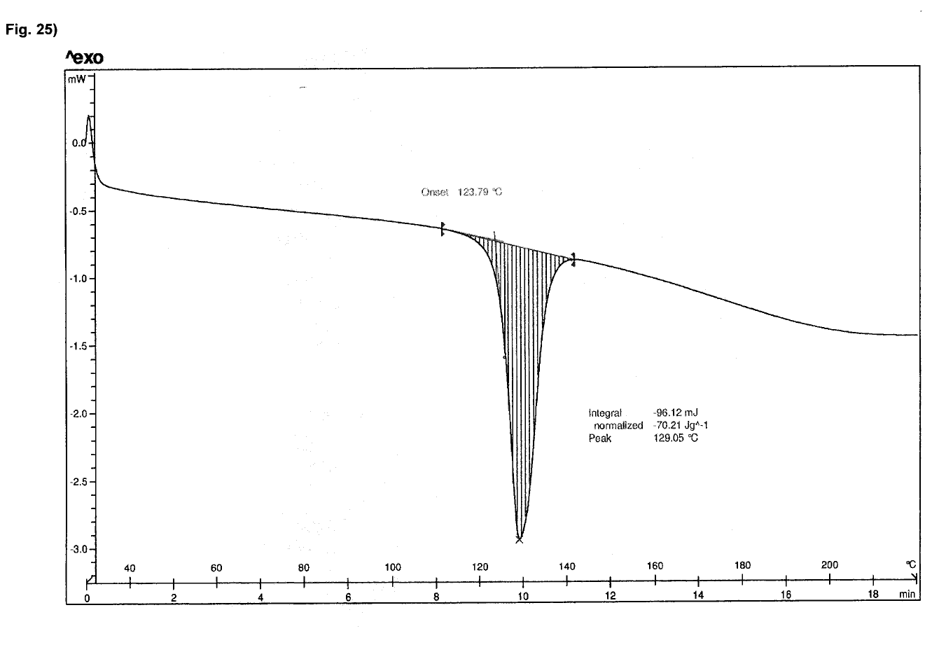

The novel type of crystal of the present invention is characterized in that the endothermic sharp peak corresponding to the melting point has an onset at 123.8° C. (fusion enthalpy −70.21 J/g), measured by DSC analysis (10° C./min) (see FIG. 25).

### TG

Thermogravimetric analysis was recorded with a thermogravimetric analyzer Mettler TGA/SDTA851^{e}. A sample of 3.2388 mg was weighed into a 70 μL alumina crucible with a pinhole lid, and was heated at 10° C./min from 30 to 200° C., under nitrogen (50 mL/min).

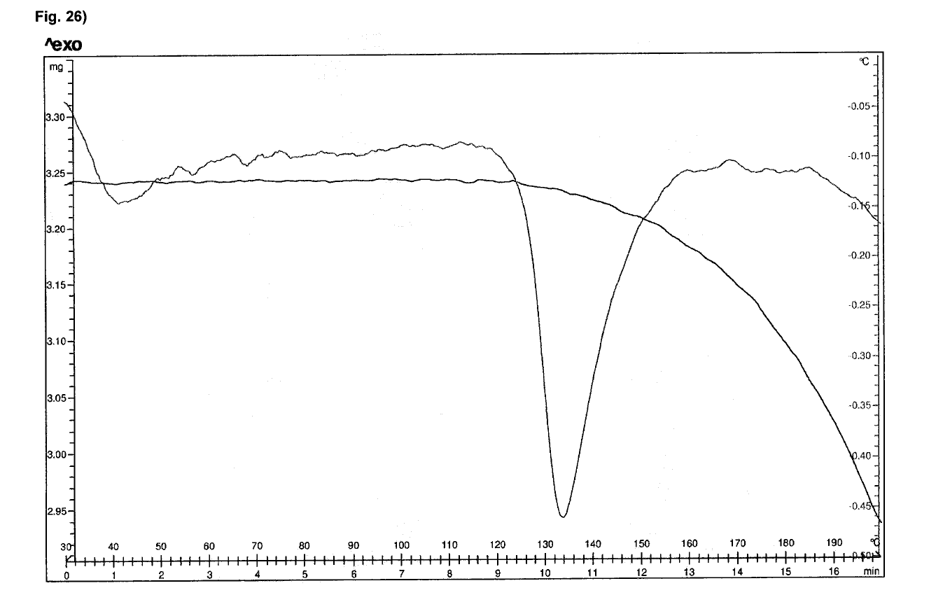

The TG analysis of crystalline this form according to the invention shows no weight loss at temperatures lower than the melting point (see FIG. 26).

### XRPD

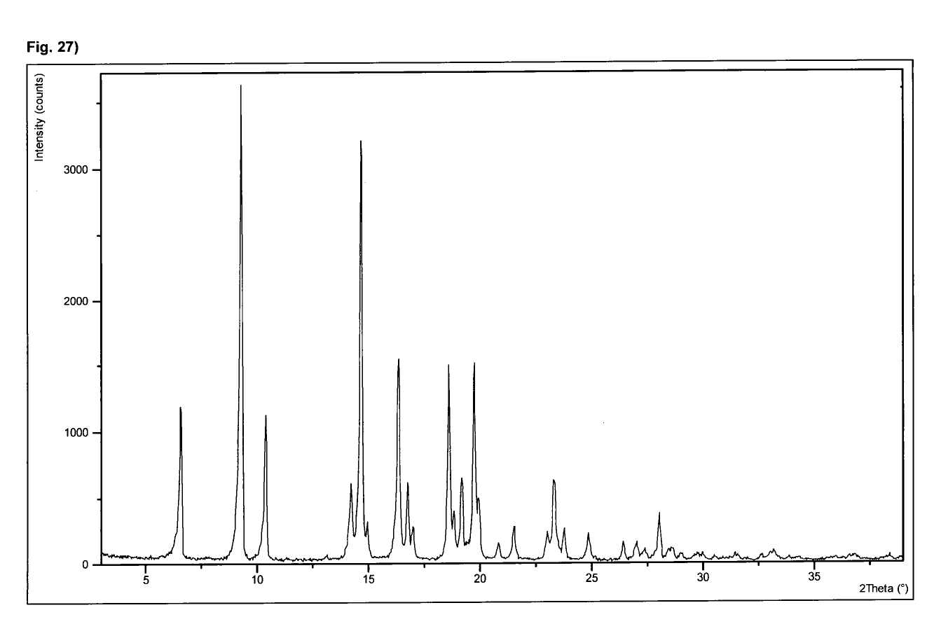

XRPD analysis was performed using a Philips X'Pert diffractometer with Cu K_{α} radiation in Bragg-Brentano geometry. The system is equipped with a proportional detector. The measurement parameters were as follows: the range of 2θ was 3° to 39° at a scan rate of 1.8° per minute (see FIG. 27).

List of Selected Peaks:

| 2θ (°) | d (Å) | I (%) |
|---|---|---|
| 6.60 | 13.40 | 32 |
| 9.30 | 9.50 | 100 |
| 10.40 | 8.50 | 29 |
| 14.23 | 6.23 | 16 |
| 14.70 | 6.03 | 88 |
| 14.97 | 5.92 | 8 |
| 16.36 | 5.42 | 42 |
| 16.77 | 5.29 | 16 |
| 17.02 | 5.21 | 7 |
| 18.61 | 4.77 | 40 |
| 18.84 | 4.71 | 10 |
| 19.18 | 4.63 | 17 |
| 19.75 | 4.50 | 41 |
| 20.83 | 4.26 | 4 |
| 21.53 | 4.13 | 7 |
| 23.01 | 3.87 | 6 |
| 23.32 | 3.82 | 17 |
| 23.77 | 3.74 | 7 |
| 24.85 | 3.58 | 6 |
| 26.42 | 3.37 | 4 |
| 27.03 | 3.30 | 3 |
| 27.40 | 3.26 | 3 |
| 28.04 | 3.18 | 10 |
| 28.60 | 3.12 | 2 |
| 29.02 | 3.08 | 1 |

### Single Crystal X-Ray Diffraction

This crystal structure has been determined from single crystal X-ray diffraction data. The colourless prismatic crystal used (0.38×0.15×0.06 mm) was obtained from the evaporation of a solution in MIK of equimolar amounts of Memantine and (rac)-Flurbiprofen.

Analysis was performed at room temperature using an Oxford Diffraction Xcalibur Gemini diffractometer with Cu K_{α} radiation equipped with a CCD detector. The intensities were measured using the oscillation method (program used: CrysAlis CCD 1.171.32.5). No significant decay of standard intensities was observed. Data reduction (Lorentz and polarization corrections) and absorption correction were applied (program used: CrysAlis RED 1.171.32.5). The structure was solved with direct methods and full-matrix least-squares refinement of Fₒ² was carried out (programs used: SIR2006 and SHELXL97). All non-hydrogen atoms were refined with anisotropic displacement parameters.

Relevant Structural Data:

| Crystal system | Orthorhombic |
|---|---|
| Space group | P2₁2₁2₁ |
| a (Å) | 6.6095(1) |
| b (Å) | 19.0963(2) |
| c (Å) | 19.1204(3) |
| Volume (Å³) | 2413.3(2) |
| Z | 4 |
| D calc. (Mg/m³) | 1.166 |
| N. of refl. | 3339 |
| Refl. with I > 2σ(I) | 2911 |
| R (I > 2σ(I)) | 0.0352 |

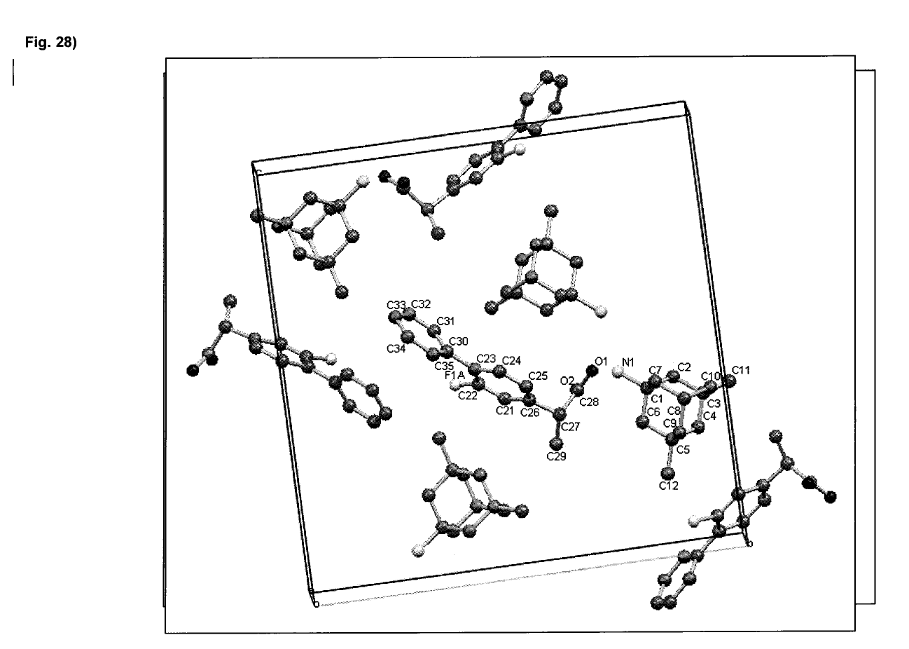

The unit cell contents of this form are depicted in FIG. 28 (hydrogen atoms have been omitted for clarity; fluorine atom is disordered over the two chemically equivalent sites and only the one with higher occupation is showed; program used: Mercury 1.4.2).

Positions of peaks in XRPD diffractogram simulated from single crystal data are almost identical to those in the experimental one presented above.

### Example 8

### Form B: Memantine-(rac)-Flurbiprofen (1:1)

### Example 8a

### Memantine-(rac)-Flurbiprofen Salt Form B

To a vial containing (rac)-Flurbiprofen (50 mg, 0.20 mmol) in solution with 1 mL AcOEt, was added Memantine (36 mg, 0.20 mmol, 1 eq.) diluted with AcOEt (1 mL) at room temperature.

The solution was evaporated slowly without stirring at room temperature under atmospheric pressure. After complete evaporation, salt form B Memantine-(rac)-Flurbiprofen 1:1 was obtained as small needle crystals (86 mg, quantitative yield). This experiment has a low reproducibility, but the sample of this product was used to seed other experiments.

### Example 8b

### Memantine-(rac)-Flurbiprofen Salt Form B

To an assay tube containing (rac)-Flurbiprofen (50 mg, 0.20 mmol) suspended in cyclohexane (0.3 mL), was added at room temperature Memantine (36 mg, 0.20 mmol, 1 eq.) diluted with cyclohexane (0.7 mL). A complete dissolution was obtained, and a seeding of form B was added. Then, the mixture is stirred 1 h to room temperature.

The white solid was filtered with a sintered funnel (porosity 3) and was washed with cyclohexane (0.3 mL). After drying at room temperature under vacuum, salt form B Memantine-(rac)-Flurbiprofen 1:1 was obtained as a white powder (46 mg, 53% yield).

### ¹H NMR

Proton nuclear magnetic resonance analyses were recorded in deuterated chloroform (CDCl₃) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum in CDCl₃ at 400 MHz shows peaks identical to those of form A.

### IR

The FTIR spectrum was recorded using a Thermo Nicolet Nexus 870 FT-IR, equipped with a beamsplitter KBr system, a 35 mW He—Ne laser as the excitation source and a DIGS KBr detector. The spectra were acquired in 32 scans at a resolution of 4 cm⁻¹.

The sample (KBr pellets) shows a Fourier Transform Infra Red spectrum with absorption bands at 2949.4 (m), 2915.8 (m), 2845.5 (m), 2645.5 (m), 1635.3 (m), 1556.5 (s), 1482.9 (m), 1455.1 (m), 1416.7 (s), 1377.3 (s), 1357.5 (s), 1319.1 (m), 1264.3 (m), 1130.1 (m), 925.6(m), 766.3 (m), 726.1 (m), 697.8 (s).

### DSC

DSC analysis was recorded with a Mettler DSC822^{e}. A sample of 1.7190 mg was weighed into a 40 μL aluminium crucible with a pinhole lid and was heated, under nitrogen (50 mL/min), at 10° C./min from 30 to 220° C.

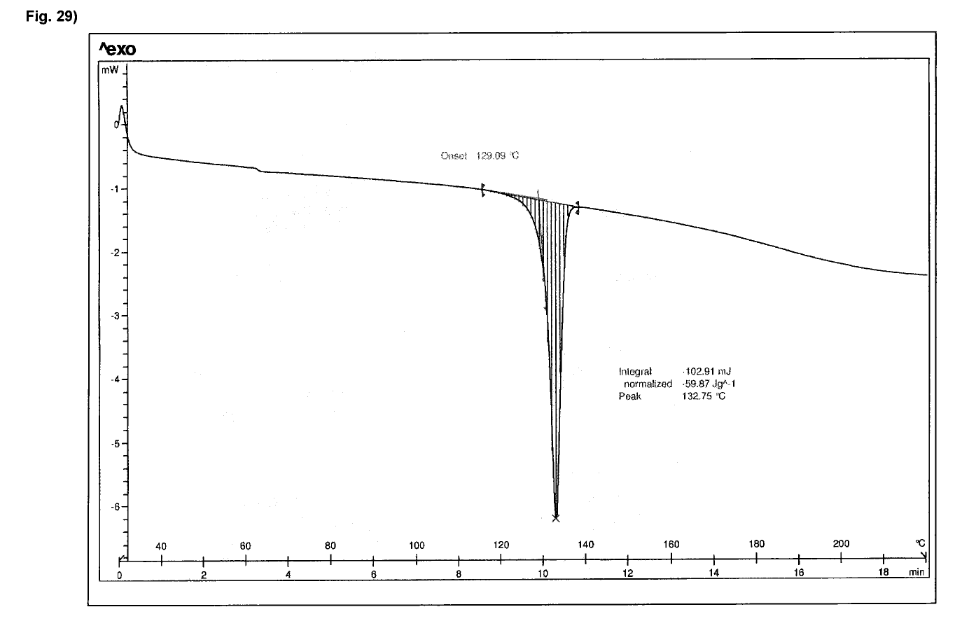

The novel type of crystal of the present invention (form B) is characterized in that the endothermic sharp peak corresponding to the melting point has an onset at 129.1° C. (fusion enthalpy −59.9 J/g), measured by DSC analysis (10° C./min) (see FIG. 29).

### TG

Thermogravimetric analysis was recorded with a thermogravimetric analyzer Mettler TGA/SDTA851^{e}. A sample of 3.8140 mg was weighed into a 70 μL alumina crucible with a pinhole lid, and was heated at 10° C./min from 30 to 200° C., under nitrogen (50 mL/min).

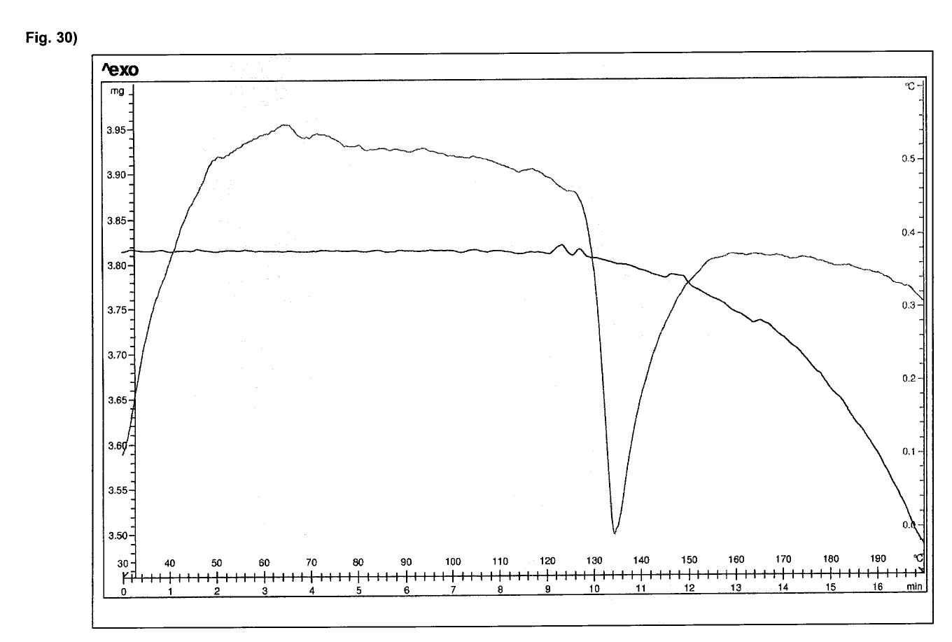

The TG analysis of crystalline form B according to the invention shows no weight loss at temperatures lower than the melting point (see FIG. 30).

### XRPD

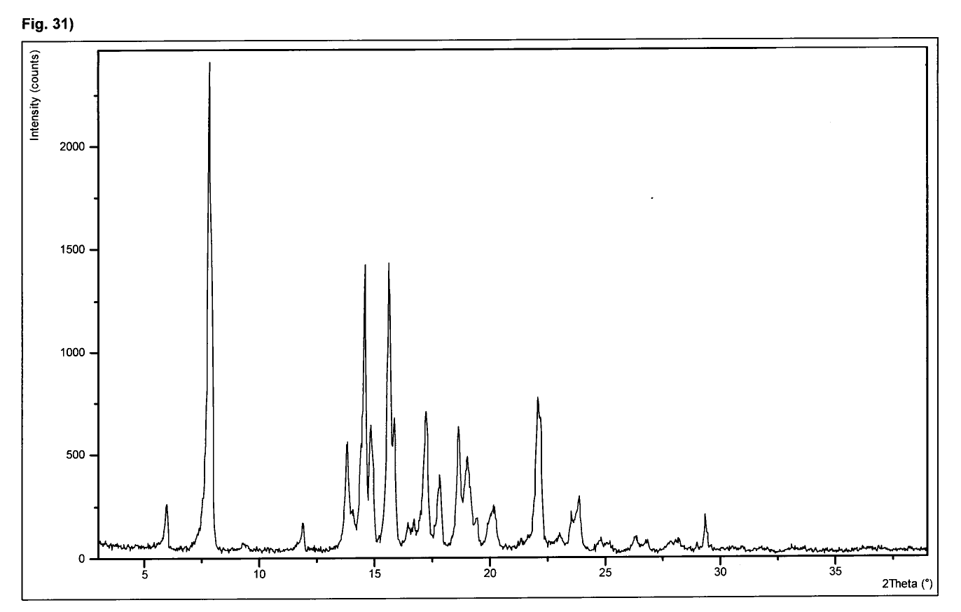

XRPD analysis was performed using a Philips X'Pert diffractometer with Cu K_{α} radiation in Bragg-Brentano geometry. The system is equipped with a proportional detector. The measurement parameters were as follows: the range of 2θ was 3° to 39° at a scan rate of 1.8° per minute (see FIG. 31).

List of Selected Peaks:

| 2θ (°) | d (Å) | I (%) |
|---|---|---|
| 5.94 | 14.87 | 10 |
| 7.85 | 11.27 | 100 |
| 9.28 | 9.53 | 2 |
| 11.89 | 7.44 | 8 |
| 13.80 | 6.42 | 25 |
| 14.53 | 6.10 | 45 |
| 14.85 | 5.97 | 25 |
| 15.62 | 5.67 | 62 |
| 16.50 | 5.37 | 6 |
| 17.23 | 5.15 | 34 |
| 17.81 | 4.98 | 14 |
| 18.68 | 4.75 | 22 |
| 20.10 | 4.42 | 12 |
| 22.06 | 4.03 | 29 |
| 23.77 | 3.74 | 11 |
| 24.92 | 3.57 | 2 |
| 26.32 | 3.39 | 2 |
| 28.03 | 3.18 | 2 |
| 29.28 | 3.06 | 3 |

### Example 9

### Form C: Memantine-(rac)-Flurbiprofen (1:1)

### Example 9a

### Memantine-(rac)-Flurbiprofen Salt Form C

To a vial containing (rac)-Flurbiprofen (50 mg, 0.20 mmol) in solution with 1 mL dioxane, was added Memantine (36 mg, 0.20 mmol, 1 eq.) diluted with dioxane (1 mL).

The solution was evaporated slowly without stirring at room temperature under atmospheric pressure. After complete evaporation, salt form C Memantine-(rac)-Flurbiprofen 1:1 was obtained as a white solid (86 mg, quantitative yield). This experiment has a low reproducibility, but the sample of this product was used to seed other experiments.

### Example 9b

### Memantine-(rac)-Flurbiprofen Salt Form C

To an assay tube containing (rac)-Flurbiprofen (50 mg, 0.20 mmol) suspended in cyclohexane (0.3 mL), was added at room temperature Memantine (36 mg, 0.20 mmol, 1 eq.) diluted with cyclohexane (0.7 mL). A complete dissolution was obtained, and a seeding of form C was added. Then, the mixture was stirred overnight at room temperature.

The white solid was filtered with a sintered funnel (porosity 3) and was washed with cyclohexane (0.3 mL). After drying at room temperature under vacuum, salt form C Memantine-(rac)-Flurbiprofen 1:1 was obtained as a white powder (42 mg, 49% yield).

### ¹H NMR

Proton nuclear magnetic resonance analyses were recorded in deuterated chloroform (CDCl₃) in a Varian Mercury 400 spectrometer, equipped with a broadband probe ATB 1H/19F/X of 5 mm. Spectra were acquired dissolving 5-10 mg of sample in 0.6 mL of deuterated solvent.

¹H NMR spectrum in CDCl₃ at 400 MHz shows peaks identical to those of form A.

### IR

The FTIR spectrum was recorded using a Thermo Nicolet Nexus 870 FT-IR, equipped with a beamsplitter KBr system, a 35 mW He—Ne laser as the excitation source and a DTGS KBr detector. The spectra were acquired in 32 scans at a resolution of 4 cm⁻¹.

The sample (KBr pellets) shows a Fourier Transform Infra Red spectrum with absorption bands at 2916.3 (m), 2636.6 (m), 1624.5 (s), 1553.3 (s), 1483.0 (s), 1456.0 (s), 1416.2 (s), 1380.0 (s), 1355.2 (s), 1127.8 (m), 925.7 (s), 874.1 (m), 765.4 (s), 697.8 (s).

### DSC

DSC analysis was recorded with a Mettler DSC822^{e}. A sample of 1.7480 mg was weighed into a 40 μL aluminium crucible with a pinhole lid and was heated, under nitrogen (50 mL/min), at 10° C./min from 30 to 150° C.

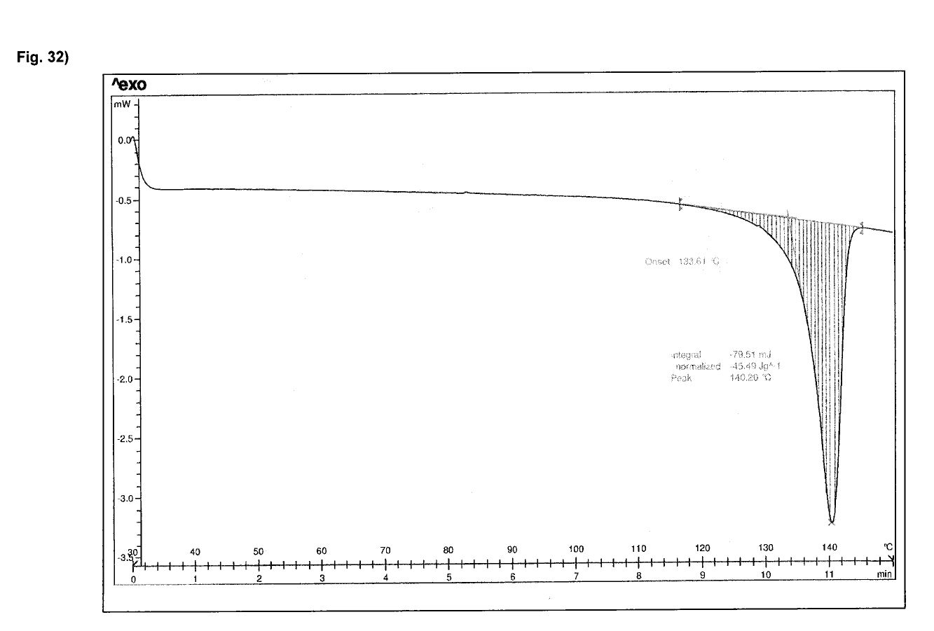

The novel type of crystal of the present invention (form C) is characterized in that the endothermic sharp peak corresponding to the melting point has an onset at 133.6° C. (fusion enthalpy −45.5 J/g), measured by DSC analysis (10° C./min), see FIG. 32.

### TG

Thermogravimetric analysis was recorded with a thermogravimetric analyzer Mettler TGA/SDTA851^{e}. A sample of 4.2325 mg was weighed into a 70 μL alumina crucible with a pinhole lid, and was heated at 10° C./min from 30 to 200° C., under nitrogen (50 mL/min).

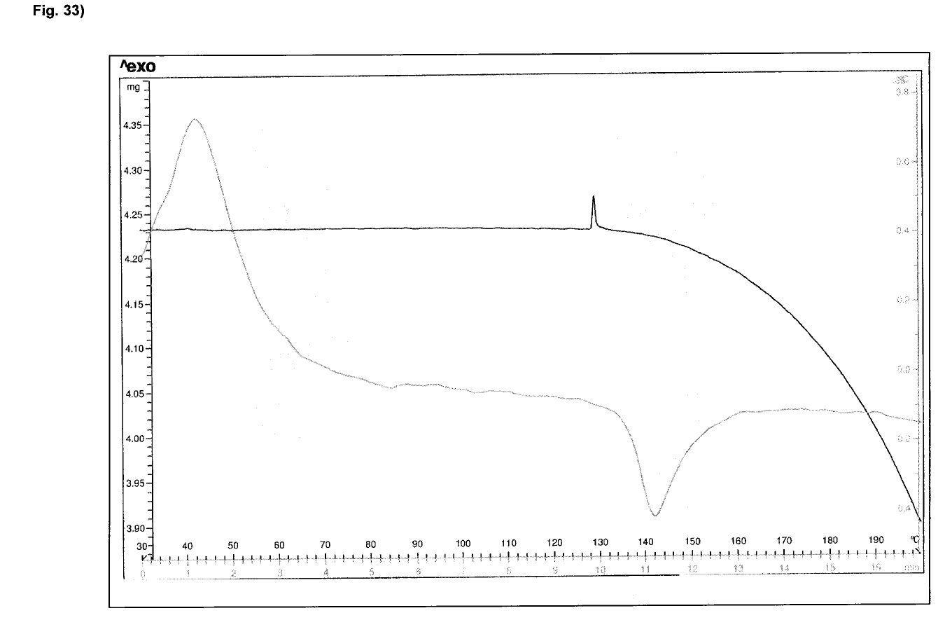

The TG analysis of crystalline form C according to the invention shows no weight loss at temperatures lower than the melting point (see FIG. 33).

### XRPD

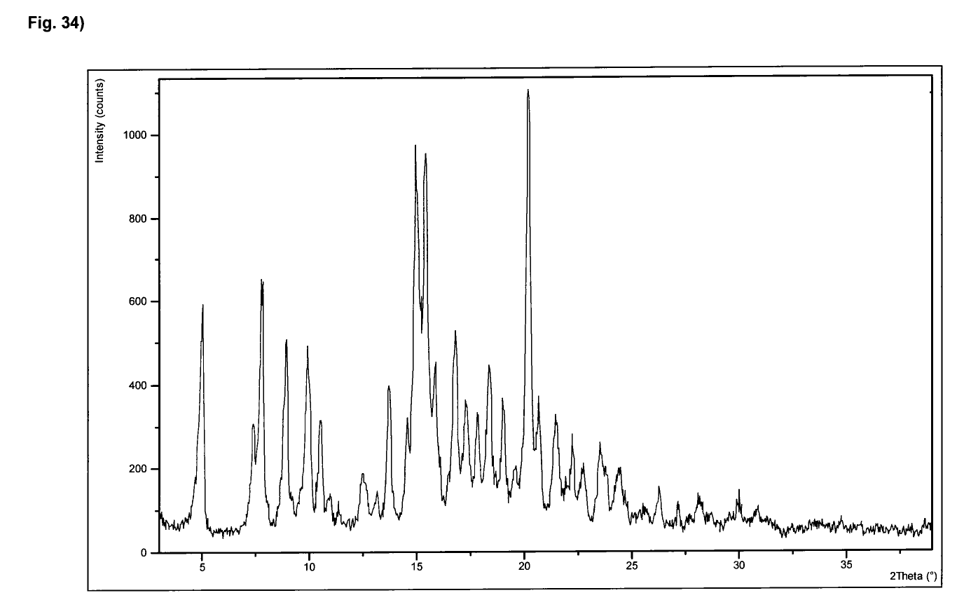

XRPD analysis was performed using a Philips X'Pert diffractometer with Cu K_{α} radiation in Bragg-Brentano geometry. The system is equipped with a proportional detector. The measurement parameters were as follows: the range of 2θ was 3° to 39° at a scan rate of 1.8° per minute (see FIG. 34).

List of Selected Peaks:

| 2θ (°) | d (Å) | I (%) |
|---|---|---|
| 5.04 | 17.52 | 53 |
| 7.39 | 11.95 | 25 |
| 7.83 | 11.29 | 58 |
| 8.96 | 9.87 | 44 |
| 9.94 | 8.90 | 42 |
| 10.54 | 8.39 | 25 |
| 10.94 | 8.08 | 8 |
| 12.46 | 7.10 | 12 |
| 13.13 | 6.74 | 8 |
| 13.74 | 6.44 | 33 |
| 15.00 | 5.91 | 86 |
| 15.43 | 5.74 | 86 |
| 15.88 | 5.58 | 39 |
| 16.84 | 5.27 | 44 |
| 17.31 | 5.12 | 29 |
| 17.84 | 4.97 | 25 |
| 18.42 | 4.82 | 35 |
| 19.01 | 4.67 | 30 |
| 20.23 | 4.39 | 100 |
| 20.70 | 4.29 | 28 |
| 21.48 | 4.14 | 25 |
| 22.25 | 4.00 | 18 |
| 22.76 | 3.91 | 13 |
| 23.48 | 3.79 | 16 |
| 24.39 | 3.65 | 12 |
| 26.28 | 3.39 | 8 |
| 27.15 | 3.28 | 5 |
| 28.20 | 3.17 | 5 |
| 30.00 | 2.98 | 5 |

1. A salt of Memantine with a COX-INHIBITOR, wherein the COX-INHIBITOR has a carboxylic group.

2. The salt according to claim 1, wherein the COX-INHIBITOR is selected from:
Acetylsalicylic acid;
Triflusal;
HTB (2-hydroxy-4-trifluoromethyl benzoic acid);
Diflunisal;
Meclofenamic acid;
Mefenamic acid;
Niflumic acid;
Flufenamic acid.
Diclofenac;
Lonazolac;
Acemetacin;
Indomethacin;
Tolmetin;
Sulindac
Etodolac;
Keterolac
Flurbiprofen;
(RS)-Flurbiprofen;
Esflurbiprofen;
(R)-Flurbiprofen;
Ibuprofen;
(RS)-Ibuprofen;
S-(+)-Ibuprofen;
R-(−)-Ibuprofen;
Ketoprofen;
(rac)-Ketoprofen
R-(−)-Ketoprofen
S-(+)-Ketoprofen
Bermoprofen;
Pelubiprofen;
Tenosal;
Aceneuramic acid;
Pirazolac;
Xinoprofen;
Flobufen;
Anirolac;
Zoliprofen;
Bromfenac;
Pemedolac;
Dexpemedolac;
Bindarit;
Romazarit;
Naproxen;
(S)-Naproxen;
(R)-Naproxen;
Tiaprofenic acid;
Ketorolac;
Fenbufen;
Fenoprofen;
Flobufen; and
Oxaprozin.

3. The salt according to claim 1, wherein the COX-INHIBITOR is selected from:
A Salicylate,
An Anthranilate,
An Arylacetic acid/Arylalkanoic acid, and
An Arylpropionic acid.

4. The salt according to claim 3, wherein the Salicylate is selected from:
Acetylsalicylic acid;
Triflusal;
HTB (2-hydroxy-4-trifluoromethyl benzoic acid); and
Diflunisal.

5. The salt according to claim 3, wherein the Anthranilate is selected from:
Meclofenamic acid;
Mefenamic acid;
Niflumic acid; and
Flufenamic acid.

6. The salt according to claim 3, wherein the Arylacetic Acid/Arylalkanoic acid is selected from:
Diclofenac;
Lonazolac;
Acemetacin;
Indomethacin;
Tolmetin;
Sulindac;
Etodolac; and
Keterolac.

7. The salt according to claim 3, wherein the Arylpropionic Acid is selected from:
Flurbiprofen;
(RS)-Flurbiprofen;
Esflurbiprofen;
(R)-Flurbiprofen;
Ibuprofen;
(RS)-Ibuprofen;
S-(+)-Ibuprofen;
R-(−)-Ibuprofen;
Ketoprofen;
(rac)-Ketoprofen;
R-(−)-Ketoprofen;
S-(+)-Ketoprofen;
Naproxen;
(S)-Naproxen;
(R)-Naproxen;
Tiaprofenic acid;
Ketorolac;
Fenbufen;
Fenoprofen;
Flobufen;
Oxaprozin;
Tolmetin;
Xinoprofen;
Flobufen;
Zoliprofen;
Bermoprofen; and
Pelubiprofen.

8. A salt of Memantine with an COX-INHIBITOR according to claim 1 selected from Memantine-Ibuprofen salt, Memantine-Flurbiprofen salt, Memantine-Diclofenac salt, Memantine-Acetylsalicylic Acid salt, Memantine-(S)-Naproxen salt, Memantine/Triflusal salt, and Memantine/2-hydroxy-4-trifluoromethyl benzoic acid (HTB) salt.

9. Crystalline form of a salt according to claim 1.

10. Process for the production of a salt according to claim 1 comprising the steps of:
dissolving an COX-INHIBITOR with a carboxylic group either as a free acid or as a salt together with, or after, or before, Memantine either as a free base or as a salt in an organic solvent,
stirring the mixture obtained at a temperature between 0° C. and 80° C.,
filtering the obtained solid and/or evaporating the solvent, and
drying of the resulting product.

11. Process according to claim 10, wherein
the organic solvent is selected from acetone, acetonitrile, isobutyl acetate, heptane, methanol, tetrahydrofuran, isopropanol, ethanol or cyclohexane; and/or
the solvent is evaporated under high vacuum; and/or
the ratio of Memantine to COX-INHIBITOR is 1:1 to 2:1, preferably 1:1; and/or
the Memantine dissolved is a free base.

12. A composition comprising at least one salt according to claim 1 and optionally one or more pharmaceutically acceptable excipients.

13. Pharmaceutical composition comprising a therapeutically effective amount of the crystalline form of a salt according to claim 1, in a physiologically acceptable medium.

14. A method for the treatment of pain, wherein said method comprises administering to a subject in need a therapeutically effective amount of at least one salt according to claim 1, and optionally one or more pharmaceutically acceptable excipients.

15. Crystalline form of a Memantine-(S)-Naproxen salt according to claim 9.

16. Crystalline form of a Memantine/Triflusal salt according to claim 9.

17. Crystalline form of a Memantine/HTB salt according to claim 9.

18. Crystalline form of a Memantine-(S)-Ibuprofen salt according to claim 9.

19. Crystalline form of a Memantine-Diclofenac salt according to claim 9.

20. Crystalline form of a Memantine-Acetylsalicylic acid salt according to claim 9.

21. Crystalline form of a Memantine-Flurbiprofen salt according to claim 9.

22. Crystalline form according to claim 21, wherein said form crystallizes as Memantine-(R)-Flurbiprofen (1:1).

23. Crystalline form according to claim 21, wherein said form crystallizes as Memantine-(RS)-Flurbiprofen (1:1).

24. The salt according to claim 6, wherein the Arylacetic Acid/Arylalkanoic acid is Diclofenac.
